(19) United States
(12) Patent Application Publication
(10) Pub. No.: US 2006/0089398 A1
(43) Pub. Date: Apr. 27, 2006

(54) Isoxazole carboxamide derivatives as ghrelin receptor modulators

(76) Inventors: Gang Liu, Gurnee, IL (US); Hongyu Zhao, Buffalo Grove, IL (US); Michael D. Serby, Mundelein, IL (US); Bo Liu, Libertyville, IL (US); Zhili Xin, Lake Bluff, IL (US); Lissa T.J. Nelson, Highland Park, IL (US); Bruce G. Szczepankiewicz, Lindenhurst, IL (US); Hing L. Sham, Vernon Hills, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 10/797,734

(22) Filed: Mar. 10, 2004

Related U.S. Application Data
(60) Provisional application No. 60/455,968, filed on Mar. 19, 2003.

(51) Int. Cl. A61K 31/42; C07D 261/18

(52) U.S. Cl. 514/378; 548/248

(57) ABSTRACT

The present invention is related to compounds of formula (I), 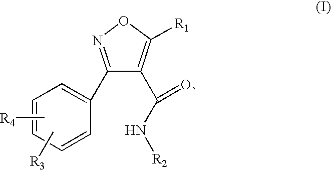 or a therapeutically suitable salt or prodrug thereof, the preparation of the compounds, compositions containing the compounds and the use of the compounds in the prevention or treatment of disorders regulated by ghrelin including anorexia, cancer cachexia, eating disorders, age-related decline in body composition, weight gain, obesity, and diabetes mellitus.

This application claims priority to the provisional application Ser. No. 60/455,968 filed on Mar. 19, 2003.

### TECHNICAL FIELD

The present invention is directed to compounds that are modulators of the ghrelin receptor, the preparation of the compounds, compositions containing the compounds and the use of the compounds in the prevention or treatment of disorders regulated by ghrelin including anorexia, cancer cachexia, eating disorders, age-related decline in body composition, weight gain, obesity, and diabetes mellitus.

### BACKGROUND OF THE INVENTION

Stimulation of food intake is important in connection with patients suffering from anorexia due to chronic medical conditions, eating disorders, and other conditions in which excessive weight loss has produced a detrimental effect on the patients' health.

Obesity is a common and very serious public health problem as it increases a person's risk for a number of serious conditions, including diabetes, heart disease, stroke, high blood pressure, and some types of cancers. Considerable increase in the number of obese individuals over the past two decades has created profound public health implications. Although studies have demonstrated that reduction in obesity by diet and exercise reduces the associated risk factors dramatically, these treatments are largely unsuccessful considering obesity is strongly associated with genetically inherited factors that contribute to increased appetite, preferences for highly caloric foods, reduced physical activity, and increased lipogenic metabolism.

Growth hormone (GH) is not only of importance for linear body growth but is also of major importance for the maintenance of body composition, metabolism and heart function in adult life. GH release from the anterior pituitary is regulated by the stimulatory peptide GH-releasing hormone (GHRH) and the inhibitory peptide somatostatin [Frohman, L; Jansson, J.-O. Endocr. Rev. (1986) 7:223-253]. Early research identified small GH-releasing peptides (GHRPs) derived from the pentapeptide met-enkephalin [Momany, F; Browers, C, et al: Endocrinology (1981) 108:31-39]. Further efforts led to the development of a number of peptidyl and non-peptidyl growth hormone secrectgogues (GHSs), including the orally-active, non-peptidyl GH secretagogue MK677 [Svensson, J; Lohn, L; Jansson, J.-O. et al: J. Clin. Endocrinol. Metab. (1998) 83:362-369]. Later efforts cloned a seven-transmembrane GPCR that was a target for the GHSs [Howard, A; Feighner, S.; Cully, D. et al: Science (1996) 273:974-977].

This GHS-receptor (GHS-R) is localized in the hypothalamus and in the pituitary, but also in other brain areas such as the hippocampus as well as the pancreas. Recently, an endogenous ligand for the GHS-R, ghrelin, an acylated peptide consisting of 28 amino acids was isolated [Kojima, M; Hosoda, H; Date, Y; Nakazoto, M.; Matsuo, H; Kangawa, K: Nature (1999), 402:656-660]. Since then, ghrelin has been found to be localized in the hypothalamic-pituitary area where it stimulates the release of GH to the circulation, but is also found in the highest concentration in the stomach.

Biological evidence indicates that ghrelin has an important role in the regulation of metabolism and energy expenditure. Ghrelin was found to stimulate food intake and weight gain when administered either systemically or intraventricularly in rodents [Nakazato M, Murakami N, Date Y, Kojima M, Matsuo H, Kangawa K, et al. Nature 2001;409:194-198] [Asakawa A, Inui A, Kaga T, Yuzuriha H, Nagata T, Ueno N, et al. Gastroenterology 2001;120:337-345]. Ghrelin was also found to be more potent than any other orexigenic peptide except neuropeptide Y (NPY). The orexigenic activity of centrally administered ghrelin is thought to be mediated by brain NPY and AGRP, two neuropeptides with potent orexigenic actions [Kamegai J, Tamura H, Shimizu T, Ishii S, Sugihara H, Wakabayashi I. Endocrinology 2000;141:4797-4800]. It was also recognized that the appetite activity of centrally administered ghrelin may be blocked by co administration of a NPY-Y1 receptor antagonist. In addition, ghrelin was found to reverse leptin-induced inhibition of food intake [Shintani M, Ogawa Y, Ebihara K, Aizawa-Abe M, Miyanaga F, Takaya K, et al. Diabetes 2001;50:227-232]. Ghrelin exerts its actions in the arcuate nucleus and paraventricular nucleus to influence the interplay of NPY, AGRP and a-MSH circuits. Ghrelin may also act via afferent vagal pathways that terminate in the hypothalamus. In obese patients, the increase in the plasma ghrelin level with diet-induced weight loss is consistent with the hypothesis that ghrelin has a role in the long-term regulation of body weight. Gastric bypass in obese patients is associated with markedly suppressed ghrelin levels, possibly contributing to the weight-reducing effect of the procedure (Cummings, D. E. et al: N Engl J Med 2002;346: 1623-30).

Intracerebroventricular treatment with the anti-ghrelin antiserum against the N-terminal region twice a day for 5 days in rats decreased significantly both daily food intake and body weight (Murakami, N; T Hayashida, T; T Kuroiwa, T; K Nakahara, K; Ida, T; Mondal, M S; Nakazato, M; Kojima M; Kangawa, K. Journal of Endocrinology (2002) 174, 283-288). Transgenic (Tg) rats expressing an antisense ghrelin receptor mRNA under the control of the promoter for tyrosine hydroxylase (TH) selectively attenuated ghrelin receptor protein expression in the arcuate nucleus (Arc). Tg rats had lower body weight and less adipose tissue than did control rats. Daily food intake was reduced, and the stimulatory effect of GHS treatment on feeding was abolished in Tg rats [Shuto, Y; Shibasaki, T; Otagiri, A; et al: J. Clin. Invest. 109:1429-1436 (2002)]. These data suggest that ghrelin receptor modulators may be beneficial in the treatment of anorexia, cancer cachexia, eating disorders, age-related decline in body composition, weight gain, obesity and disorders associated with obesity such as diabetes mellitus.

### SUMMARY OF THE INVENTION

The present invention is directed to compounds of formula (I), 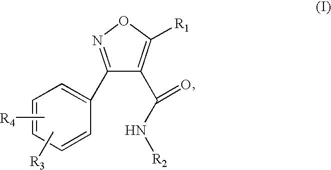
or a therapeutically suitable salt or prodrug thereof, wherein

R₁ is a member selected from the group consisting of alkoxyalkyl, alkyl, alkylC(O)NHalkyl, alkylS(O)₂NHalkyl, alkenyl, aryl, arylalkyl, heterocycle, heterocyclealkyl, hydroxyalkyl, RₐR_{b}N—, RₐR_{b}Nalkyl, RₐR_{b}Ncarboxyalkyl, wherein the alkyl group of said arylalkyl and the alkyl group of said heterocyclealkyl may be substituted with 0, 1 or 2 groups that are a member selected from the group consisting of halogen and hydroxy;

R₂ is a member selected from the group consisting of alkyl, aryl, arylalkyl, cycloalkyl, cycloalkylalkyl, cycloalkenyl, cycloalkenylalkyl;

R₃ and R₄ are each members independently selected from the group consisting of hydrogen, alkyl, alkoxy, aryl, halogen, haloalkyl, cycloalkyl, cyano and nitro;

Rₐ and R_{b} are each members independently selected from the group consisting of hydrogen, alkoxyalkyl, alkyl, alkylcarbonyl, alkylsulfonyl, aryloxyalkyl and R_{c}R_{d}Ncarboxyalkylcarbonyl; and

R_{c} and R_{d} are each members independently selected from the group consisting of hydrogen, and alkyl.

According to one embodiment of the present invention there is provided a method of treating disorders regulated by ghrelin including obesity, eating disorders, weight gain and diabetes mellitus, in a mammal.

According to still another embodiment, the present invention is directed to a pharmaceutical composition comprising a therapeutically effective amount of a compound of formula (I) in combination with a pharmaceutically suitable carrier.

### DETAILED DESCRIPTION OF THE INVENTION

### DEFINITIONS

As used throughout this specification and the appended claims, the following terms have the following meanings:

The term “alkenyl,” as used herein, refers to a straight or branched chain hydrocarbon containing from 2 to 10 carbons and containing at least one carbon-carbon double bond formed by the removal of two hydrogens. The term “alkenyl,” as related to the compounds of the present invention, refer to C₂-alkenyl, C₃-alkenyl, C₄-alkenyl, C₅-alkenyl, C₆-alkenyl, C₇-alkenyl, C₈-alkenyl, C₉-alkenyl or C₁₀-alkenyl or any combination thereof. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-i-heptenyl, and 3-decenyl.

The term “alkoxy,” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxyalkyl,” as used herein, refers to an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, and methoxymethyl.

The term “alkoxycarbonyl,” as used herein, refers to an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl.

The term “alkyl,” as used herein, refers to a straight or branched chain hydrocarbon containing from 1 to 10 carbon atoms. The term “alkyl,” as related to the compounds of the present invention, refer to C₁-alkyl, C₂-alkyl, C₃-alkyl, C₄-alkyl, C₅-alkyl, C₆-alkyl, C₇-alkyl, C₈-alkyl, C₉-alkyl or C₁₀-alkyl or any combination thereof. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylcarbonyl,” as used herein, refers to an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkylsulfonyl,” as used herein, refers to an alkyl group, as defined herein, appended to the parent molecular moiety through a sulfonyl group, as defined herein. Representative examples of alkylsulfonyl include, but are not limited to, methylsulfonyl and ethylsulfonyl.

The term “alkynyl,” as used herein, refers to a straight or branched chain hydrocarbon group containing from 2 to 10 carbon atoms and containing at least one carbon-carbon triple bond. The term “alkynyl,” as related to the compounds of the present invention, refer to C₂-alkynyl, C₃-alkynyl, C₄-alkynyl, C₅-alkynyl, C₆-alkynyl, C₇-alkynyl, C₈-alkynyl, C₉-alkynyl or C₁₀-alkynyl or any combination thereof. Representative examples of alkynyl include, but are not limited, to acetylenyl, 1-propynyl, 2-propynyl, 3-butynyl, 2-pentynyl, and 1-butynyl.

The term “aryl,” as used herein, refers to a monocyclic-ring system, or a bicyclic- or a tricyclic-fused ring system wherein one or more of the fused rings are aromatic. Representative examples of aryl include, but are not limited to, anthracenyl, azulenyl, fluorenyl, indanyl, indenyl, naphthyl, phenyl, and tetrahydronaphthyl.

The aryl groups of this invention may be substituted with 0, 1, 2, or 3 substituents which are members independently selected from the group consisting of alkenyl, alkoxy, alkoxyalkyl, alkoxyalkylNHalkyl, alkoxycarbonyl, alkoxycarbonylalkyl, alkyl, alkylNHalkyl, alkylcarbonyl, alkylcarbonylalkyl, alkylcarbonyloxy, alkylsulfonyl, alkylthio, alkynyl, arylalkylNHalkyl, aryloxyalkylNHalkyl, carboxy, carboxyalkyl, cyano, cyanoalkyl, cycloalkoxyalkylNHalkyl, cycloalkylNHalkyl, cycloalkenylalkylNHalkyl, formyl, halogen, haloalkyl, heterocycle, heterocyclealkyl, heterocyclealkylNHcarbonyl, heterocyclecarbonyl, haloalkyl, hydroxy, hydroxyalkyl, mercapto, nitro, phenyl, R_{g}RⱼN— and R_{g}RⱼNalkyl- wherein R_{g} and Rⱼ are each members independently selected from the group consisting of hydrogen, alkyl, alkylcarbonyl, alkylsulfonyl, aryl, arylalkyl, cycloalkyl, cycloalkylalkyl, and heterocyclealkyl.

The term “arylalkoxy,” as used herein, refers to an aryl group, as defined herein, appended to the parent molecular moiety through an alkoxy group, as defined herein. Representative examples of arylalkoxy include, but are not limited to, 2-phenylethoxy, 3-naphth-2-ylpropoxy, and 5-phenylpentyloxy.

The term “arylalkyl,” as used herein, refers to an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

The term “arylcarbonyl,” as used herein, refers to an aryl group, as defined herein, appended to the parent molecular moiety through a carbonyl group, as defined herein. Representative examples of arylcarbonyl include, but are not limited to, benzoyl and naphthoyl.

The term “carbonyl,” as used herein, refers to a —C(O)— group.

The term “carboxy,” as used herein, refers to a —CO₂H group.

The term “carboxyalkyl,” as used herein, refers to a carboxy group, as defined herein, appended to the parent molecular moiety through an alkyl group, as defined herein. Representative examples of carboxyalkyl include, but are not limited to, carboxymethyl, 2-carboxyethyl, and 3-carboxypropyl.

The term “cyano,” as used herein, refers to a —CN group.

The term “cyanoalkyl,” as used herein, refers to a cyano group, as defined herein, appended to the parent molecular moiety through an alkyl group, as defined herein. Representative examples of cyanoalkyl include, but are not limited to, cyanomethyl, 2-cyanoethyl, and 3-cyanopropyl.

The term “cycloalkyl,” as used herein, refers to a monocyclic, bicyclic, or tricyclic ring system. Monocyclic ring systems are exemplified by a saturated cyclic hydrocarbon group containing from 3 to 8 carbon atoms. The term “cycloalkyl,” as related to the compounds of the present invention refer to C₃-cycloalkyl, C₄-cycloalkyl, C₅-cycloalkyl, C₆-cycloalkyl, C₇-cycloalkyl or C₈-cycloalkyl. Examples of monocyclic ring systems include cyclopropyl, cyclobutyl, cyclopentyl, cyclohexyl, cycloheptyl, and cyclooctyl. Bicyclic ring systems are exemplified by a bridged monocyclic ring system in which two non-adjacent carbon atoms of the monocyclic ring are linked by an alkylene bridge of between one and three additional carbon atoms. Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Tricyclic ring systems are exemplified by a bicyclic ring system in which two non-adjacent carbon atoms of the bicyclic ring are linked by a bond or an alkylene bridge of between one and three carbon atoms. Representative examples of tricyclic-ring systems include, but are not limited to, tricyclo[3.3.1 0^{3,7}]nonane and tricyclo[3.3.1.1^{3,7}]decane (adamantane).

The cycloalkyl groups of this invention may be substituted with 0, 1, 2, or 3 substituents which are members independently selected from the group consisting of alkenyl, alkoxy, alkoxyalkyl, alkoxyalkylNHalkyl, alkoxycarbonyl, alkoxycarbonylalkyl, alkyl, alkylNHalkyl, alkylcarbonyl, alkylcarbonylalkyl, alkylcarbonyloxy, alkylsulfonyl, alkylthio, alkynyl, arylalkylNHalkyl, aryloxyalkylNHalkyl, carboxy, carboxyalkyl, cyano, cyanoalkyl, cycloalkoxyalkylNHalkyl, cycloalkylNHalkyl, cycloalkenylalkylNHalkyl, formyl, halogen, haloalkyl, heterocycle, heterocyclealkyl, heterocyclealkylNHcarbonyl, heterocyclecarbonyl, hydroxy, hydroxyalkyl, mercapto, nitro, phenyl, R_{g}RⱼN- and R_{g}RⱼNalkyl- wherein R_{g} and Rⱼ are defined herein.

The term “cycloalkenyl,” as used herein, refers to a monocyclic, bicyclic, or tricyclic ring system which contains 1 or 2 double bonds by is not aromatic. Monocyclic ring systems are exemplified by an unsaturated cyclic hydrocarbon group containing from 3 to 8 carbon atoms. Examples of monocyclic ring systems include cyclopropenyl, cyclobutenyl, cyclopentenyl, cyclohexenyl, cycloheptenyl, and cyclooctenyl. Bicyclic ring systems are exemplified by a bridged monocyclic ring system in which two non-adjacent carbon atoms of the monocyclic ring are linked by an alkylene bridge of between one and three additional carbon atoms.

The cycloalkenyl groups of this invention may be substituted with 0, 1, 2, or 3 substituents which are members independently selected from the group consisting of alkenyl, alkoxy, alkoxyalkyl, alkoxyalkylNHalkyl, alkoxycarbonyl, alkoxycarbonylalkyl, alkyl, alkylNHalkyl, alkylcarbonyl, alkylcarbonylalkyl, alkylcarbonyloxy, alkylsulfonyl, alkylthio, alkynyl, arylalkylNHalkyl, aryloxyalkylNHalkyl, carboxy, carboxyalkyl, cyano, cyanoalkyl, cycloalkoxyalkylNHalkyl, cycloalkylNHalkyl, cycloalkenylalkylNHalkyl, formyl, halogen, haloalkyl, heterocycle, heterocyclealkyl, heterocyclealkylNHcarbonyl, heterocyclecarbonyl, hydroxy, hydroxyalkyl, mercapto, nitro, phenyl and R_{g}RⱼN-wherein R_{g} and Rⱼ are defined herein.

The term “formyl,” as used herein, refers to a —C(O)H group.

The term “halo” or “halogen,” as used herein, refers to —Cl, —Br, —I or —F.

The term “haloalkyl,” as used herein, refers to at least one halogen, as defined herein, appended to the parent molecular moiety through an alkyl group, as defined herein.

Representative examples of haloalkyl include, but are not limited to, chloromethyl, 2-fluoroethyl, trifluoromethyl, pentafluoroethyl, and 2-chloro-3-fluoropentyl.

The term “heterocycle” or “heterocyclic,” as used herein, refers to a monocyclic, bicyclic, or tricyclic ring system. Monocyclic ring systems are exemplified by any 3- or 4-membered ring containing a heteroatom independently a member selected from oxygen, nitrogen and sulfur; or a 5-, 6- or 7-membered ring containing one, two or three heteroatoms wherein the heteroatoms are independently a member selected from nitrogen, oxygen and sulfur. The 5-membered ring has from 0-2 double bonds and the 6- and 7-membered ring have from 0-3 double bonds. Representative examples of monocyclic ring systems include, but are not limited to, azetidinyl, azepanyl, aziridinyl, diazepinyl, 1,3-dioxolanyl, dioxanyl, dithianyl, furyl, imidazolyl, imidazolinyl, imidazolidinyl, isothiazolyl, isothiazolinyl, isothiazolidinyl, isoxazolyl, isoxazolinyl, isoxazolidinyl, morpholinyl, oxadiazolyl, oxadiazolinyl, oxadiazolidinyl, oxazolyl, oxazolinyl, oxazolidinyl, piperazinyl, piperidinonyl, piperidinyl, pyranyl, pyrazinyl, pyrazolyl, pyrazolinyl, pyrazolidinyl, pyridinyl, pyrimidinyl, pyridazinyl, pyrrolyl, pyrrolinyl, pyrrolidinyl, pyrrolidinonyl, tetrahydrofuranyl, tetrahydrothienyl, tetrazinyl, tetrazolyl, thiadiazolyl, thiadiazolinyl, thiadiazolidinyl, thiazolyl, thiazolinyl, thiazolidinyl, thienyl, thiomorpholinyl, 1,1-dioxidothiomorpholinyl (thiomorpholine sulfone), thiopyranyl, triazinyl, triazolyl, and trithianyl. Bicyclic ring systems are exemplified by any of the above monocyclic ring systems fused to an aryl group as defined herein, a cycloalkyl group as defined herein, or another monocyclic ring system. Representative examples of bicyclic ring systems include but are not limited to, for example, benzimidazolyl, benzodioxinyl, benzothiazolyl, benzothienyl, benzotriazolyl, benzoxazolyl, benzofuranyl, benzopyranyl, benzothiopyranyl, cinnolinyl, indazolyl, indolyl, 2,3-dihydroindolyl, indolizinyl, methylenebenzodioxyl, naphthyridinyl, isobenzofuranyl, isobenzothienyl, isoindolyl, isoquinolinyl, phthalazinyl, 4H-pyrido[1,2-a]pyrimidin-4-one, pyranopyridinyl, quinolinyl, quinolizinyl, quinoxalinyl, quinazolinyl, tetrahydroisoquinolinyl, tetrahydroquinolinyl, and thiopyranopyridinyl. Tricyclic rings systems are exemplified by any of the above bicyclic ring systems fused to an aryl group as defined herein, a cycloalkyl group as defined herein, or a monocyclic ring system. Representative examples of tricyclic ring systems include, but are not limited to, acridinyl, carbazolyl, carbolinyl, dibenzo[b,d]furanyl, dibenzo[b,d]thienyl, naphtho[2,3-b]furan, naphtho[2,3-b]thienyl, phenazinyl, phenothiazinyl, phenoxazinyl, thianthrenyl, thioxanthenyl and xanthenyl.

According to the present invention, heterocycles of this invention may be substituted with 0, 1, 2,or 3 substituents which are members independently selected from the group consisting of alkenyl, alkoxy, alkoxyalkyl, alkoxyalkylNHalkyl, alkoxycarbonyl, alkoxycarbonylalkyl, alkyl, alkylNHalkyl, alkylcarbonyl, alkylcarbonylalkyl, alkylcarbonyloxy, alkylsulfonyl, alkylthio, alkynyl, arylalkylNHalkyl, aryloxyalkylNHalkyl, carboxy, carboxyalkyl, cyano, cyanoalkyl, cycloalkoxyalkylNHalkyl, cycloalkylNHalkyl, cycloalkenylalkylNHalkyl, formyl, halogen, haloalkyl, heterocycle, heterocyclealkyl, heterocyclealkylNHcarbonyl, heterocyclecarbonyl, hydroxy, hydroxyalkyl, mercapto, nitro, phenyl, R_{g}RⱼN—, R_{g}RⱼNcarbonyl, and R_{g}RⱼNalkyl, wherein R_{g} and Rⱼ are defined herein.

The term “heterocyclealkyl,” as used herein, refers to a heterocycle, as defined herein, appended to the parent molecular moiety through an alkyl group, as defined herein. Representative examples of heterocyclealkyl include, but are not limited to, pyridin-3-ylmethyl and 2-pyrimidin-2-ylpropyl and the like.

The term “hydroxy,” as used herein, refers to an —OH group.

The term “hydroxyalkyl,” as used herein, refers to at least one hydroxy group, as defined herein, appended to the parent molecular moiety through an alkyl group, as defined herein. Representative examples of hydroxyalkyl include, but are not limited to, 2-hydroxyethyl, 2-hydroxypropyl, 1,2-dihydroxypropyl, 3-hydroxybutyl and the like.

Accordingly the principle embodiment of the present invention is directed to compounds of formula (I), 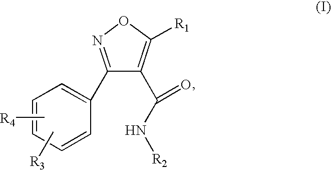
or a therapeutically suitable salt or prodrug thereof, wherein

R₁ is a member selected from the group consisting of alkoxyalkyl, alkyl, alkylC(O)NHalkyl, alkylS(O)₂NHalkyl, alkenyl, aryl, arylalkyl, heterocycle, heterocyclealkyl, hydroxyalkyl, RₐR_{b}N—, RₐR_{b}Nalkyl, and RₐR_{b}Ncarboxyalkyl, wherein the alkyl group of said arylalkyl and the alkyl group of said heterocyclealkyl may be substituted with 0, 1 or 2 groups that are a member independently selected from the group consisting of halogen and hydroxy;

R₂ is a member selected from the group consisting of alkyl, aryl, arylalkyl, cycloalkyl, cycloalkylalkyl, cycloalkenyl, cycloalkenylalkyl, heterocycle and heterocyclealkyl;

R₃ and R₄ are each members independently selected from the group consisting of hydrogen, alkyl, alkoxy, aryl, halogen, haloalkyl, cycloalkyl, cyano and nitro;

Rₐ and R_{b} are each members independently selected from the group consisting of hydrogen, alkoxyalkyl, alkyl, alkylcarbonyl, alkylsulfonyl, aryloxyalkyl and R_{c}R_{d}Ncarboxyalkylcarbonyl; and

R_{c} and R_{d} are each members independently selected from the group consisting of hydrogen, and alkyl.

According to another embodiment, the present invention is directed to compounds of formula (Ia), 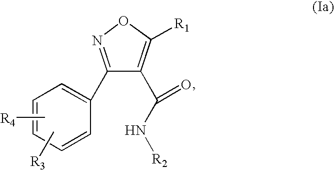
or a therapeutically suitable salt or prodrug thereof, wherein

R₁ is a member selected from the group consisting of aryl, arylalkyl, heterocycle, and heterocyclealkyl, wherein the alkyl group of said arylalkyl and the alkyl group of said heterocyclealkyl is a member selected from the group consisting of C₁-alkyl, C₂-alkyl, C₃-alkyl, C₄-alkyl, C₅-alkyl, C₆-alkyl, C₇-alkyl, C₈-alkyl, C₉-alkyl and C₁₀-alkyl and may be substituted with 0, 1 or 2 groups that are a member independently selected from the group consisting of halogen and hydroxy;

R₂ is a member selected from the group consisting of alkyl, aryl, arylalkyl, cycloalkyl, cycloalkylalkyl, cycloalkenyl, cycloalkenylalkyl, heterocycle and heterocyclealkyl;

R₃ and R₄ are each members independently selected from the group consisting of hydrogen, alkyl, alkoxy, aryl, halogen, haloalkyl, cycloalkyl, cyano and nitro.

According to a further embodiment of the present invention there is provided a compound of formula (Ia), wherein R₁ is a member selected from the group consisting of aryl, arylalkyl, heterocycle, and heterocyclealkyl, wherein the alkyl group of said arylalkyl and the alkyl group of said heterocyclealkyl is a member selected from the group consisting of C₁-alkyl, C₂-alkyl, C₃-alkyl, C₄-alkyl, C₅-alkyl, C₆-alkyl, C₇-alkyl, C₈-alkyl, C₉-alkyl and C₁₀-alkyl and may be substituted with 0, 1 or 2 groups that are a members independently selected from the group consisting of halogen and hydroxy; R₂ is a member selected from the group consisting of aryl, arylalkyl, cycloalkyl, cycloalkylalkyl, heterocycle and heterocyclealkyl; and R₃ and R₄ are each members independently selected from the group consisting of hydrogen, alkyl, halogen and nitro wherein alkyl is a member selected from the group consisting of C₁-alkyl, C₂-alkyl, C₃-alkyl, C₄-alkyl, C₅-alkyl, C6-alkyl, C₇-alkyl, C₈-alkyl, C₉-alkyl and C₁₀-alkyl.

According to a further embodiment of the present invention there is provided a compound of formula (Ia), wherein R₁ is a member selected from the group consisting of aryl, arylalkyl, heterocycle, and heterocyclealkyl, wherein the alkyl group of said arylalkyl and the alkyl group of said heterocyclealkyl is a member selected from the group consisting of C₁-alkyl, C₂-alkyl, C₃-alkyl, C₄-alkyl, C₅-alkyl, C₆-alkyl, C₇-alkyl, C₈-alkyl, C₉-alkyl or C₁₀-alkyl and may be substituted with 0, 1 or 2 groups that are a member selected from the group consisting of halogen and hydroxy; R₂ is a member selected from the group consisting of aryl, arylalkyl, heterocycle and heterocyclealkyl; and R₃ and R₄ are each independently a member selected from the group consisting of hydrogen, alkyl, halogen and nitro wherein alkyl is a member selected from the group consisting of C₁-alkyl, C₂-alkyl, C₃-alkyl, C₄-alkyl, C₅-alkyl, C₆-alkyl, C₇-alkyl, C₈-alkyl, C₉-alkyl and C₁₀-alkyl.

According to a further embodiment of the present invention there is provided a compound of formula (Ia), wherein R₁ is a member selected from the group consisting of aryl and heterocycle; R₂ is a member selected from the group consisting of aryl, arylalkyl, heterocycle and heterocyclealkyl; and R₃ and R₄ are each members independently selected from the group consisting of hydrogen, alkyl, halogen and nitro wherein alkyl is a member selected from the group consisting of C₁-alkyl, C₂-alkyl, C₃-alkyl, C₄-alkyl, C₅-alkyl, C₆-alkyl, C₇-alkyl, C₈-alkyl, C₉-alkyl and C₁₀-alkyl.

According to a further embodiment of the present invention there is provided a compound of formula (Ia), wherein R₁ is a member selected from the group consisting of arylalkyl and heterocyclealkyl; wherein the alkyl group of said arylalkyl and the alkyl group of said heterocyclealkyl is a member selected from the group consisting of C₁-alkyl, C₂-alkyl, C₃-alkyl, C₄-alkyl, C₅-alkyl, C₆-alkyl, C₇-alkyl, C₈-alkyl, C₉-alkyl and C₁₀-alkyl and may be substituted with 0, 1 or 2 groups that are a member selected from the group consisting of halogen and hydroxy; R₂ is a member selected from the group consisting of aryl, arylalkyl, heterocycle and heterocyclealkyl; and R₃ and R₄ are each members independently selected from the group consisting of hydrogen, alkyl, halogen and nitro wherein alkyl is a member selected from the group consisting of C₁-alkyl, C₂-alkyl, C₃-alkyl, C₄-alkyl, C₅-alkyl, C₆-alkyl, C₇-alkyl, C₈-alkyl, C₉-alkyl or C₁₀-alkyl.

According to another embodiment, the present invention is directed to compounds of formula (Ib), 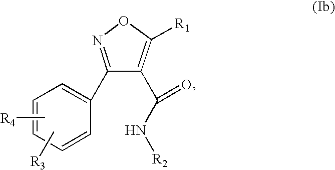
or a therapeutically suitable salt or prodrug thereof, wherein

R₁ is a member selected from the group consisting of alkoxyalkyl, alkyl, alkylC(O)NHalkyl, alkylS(O)₂NHalkyl, alkenyl, hydroxyalkyl, RₐR_{b}N—, RₐR_{b}Nalkyl, and RₐR_{b}Ncarboxyalkyl;

R₂ is a member selected from the group consisting of alkyl, aryl, arylalkyl, cycloalkyl, cycloalkylalkyl, cycloalkenyl, cycloalkenylalkyl, heterocycle and heterocyclealkyl;

R₃ and R₄ are each members independently selected from the group consisting of hydrogen, alkyl, alkoxy, aryl, halogen, haloalkyl, cycloalkyl, cyano and nitro;

Rₐ and R_{b} are each members independently selected from the group consisting of hydrogen, alkoxyalkyl, alkyl, alkylcarbonyl, alkylsulfonyl, aryloxyalkyl and R_{c}R_{d}Ncarboxyalkylcarbonyl; and

R_{c} and R_{d} are each members independently selected from the group consisting of hydrogen and alkyl;

wherein alkyl or alkyl of said alkoxyalkyl, alkylC(O)NHalkyl, alkylS(O)₂NHalkyl, hydroxyalkyl, RₐR_{b}Nalkyl, RₐR_{b}Ncarboxyalkyl, arylalkyl, cycloalkyl, cycloalkylalkyl, cycloalkenylalkyl, heterocyclealkyl are each a member independently selected from the group consisting of C₁-alkyl, C₂-alkyl, C₃-alkyl, C₄-alkyl, C₅-alkyl, C₆-alkyl, C₇-alkyl, C₈-alkyl, C₉-alkyl and C₁₀-alkyl.

According to a further embodiment of the present invention there is provided a compound of formula (Ib), wherein R₁ is a member selected from the group consisting of alkoxyalkyl, alkyl, alkylC(O)NHalkyl, alkylS(O)₂NHalkyl, alkenyl, hydroxyalkyl, RₐR_{b}N—, RₐR_{b}Nalkyl, and RₐR_{b}Ncarboxyalkyl; R₂ is a member selected from the group consisting of aryl, arylalkyl, cycloalkyl, cycloalkylalkyl, heterocycle and heterocyclealkyl; and R₃ and R₄ are each members independently selected from the group consisting of hydrogen, alkyl, halogen and nitro, wherein alkyl or alkyl of said alkoxyalkyl, alkylC(O)NHalkyl, alkylS(O)₂NHalkyl, hydroxyalkyl, RₐR_{b}Nalkyl, and RₐR_{b}Ncarboxyalkyl is a member selected from the group consisting of C₁-alkyl, C₂-alkyl, C₃-alkyl, C₄-alkyl, C₅-alkyl, C₆-alkyl, C₇-alkyl, C₈-alkyl, C₉-alkyl and C₁₀-alkyl.

According to a further embodiment of the present invention there is provided a compound of formula (Ib), wherein R₁ is a member selected from the group consisting of alkoxyalkyl, alkyl, alkylC(O)NHalkyl, alkylS(O)₂NHalkyl, alkenyl, hydroxyalkyl, RₐR_{b}N—, RₐR_{b}Nalkyl, and RₐR_{b}Ncarboxyalkyl; R₂ is a member selected from the group consisting of aryl and arylalkyl; and R₃ and R₄ are each members independently selected from the group consisting of hydrogen, alkyl, halogen and nitro, wherein alkyl or alkyl of said alkoxyalkyl, alkylC(O)NHalkyl, alkylS(O)₂NHalkyl, hydroxyalkyl, RₐR_{b}Nalkyl, and RₐR_{b}Ncarboxyalkyl is a member selected from the group consisting of C₁-alkyl, C₂-alkyl, C₃-alkyl, C₄-alkyl, C₅-alkyl, C₆-alkyl, C₇-alkyl, C₈-alkyl, C₉-alkyl and C₁₀-alkyl.

According to a further embodiment of the present invention there is provided a compound of formula (Ib), wherein R₁ is a member selected from the group consisting of alkoxyalkyl, alkyl, alkenyl and hydroxyalkyl; R₂ is a member selected from the group consisting of aryl and arylalkyl; and R₃ and R₄ are each members independently selected from the group consisting of hydrogen, alkyl, halogen and nitro, wherein alkyl or alkyl of said alkoxyalkyl, and hydroxyalkyl are member independently selected from the group consisting of C₁-alkyl, C₂-alkyl, C₃-alkyl, C₄-alkyl, C₅-alkyl, C₆-alkyl, C₇-alkyl, C₈-alkyl, C₉-alkyl or C₁₀-alkyl.

According to a further embodiment of the present invention there is provided a compound of formula (Ib), wherein R₁ is a member selected from the group consisting of alkylC(O)NHalkyl and alkylS(O)₂NHalkyl; R₂ is a member selected from the group consisting of aryl and arylalkyl; and R₃ and R₄ are eachmembers independently selected from the group consisting of hydrogen, alkyl, halogen and nitro wherein alkyl or alkyl of said alkylC(O)NHalkyl, alkylS(O)₂NHalkyl and arylalkyl are each member independently selected from the group consisting of C₁-alkyl, C₂-alkyl, C₃-alkyl, C₄-alkyl, C₅-alkyl, C₆-alkyl, C₇-alkyl, C₈-alkyl, C₉-alkyl or C₁₀-alkyl.

According to a further embodiment of the present invention there is provided a compound of formula (Ib), wherein R₁ is a member selected from the group consisting of RₐR_{b}N—, RₐR_{b}Nalkyl, and RₐR_{b}Ncarboxyalkyl; R₂ is a member selected from the group consisting of aryl and arylalkyl; and R₃ and R₄ are each members independently selected from the group consisting of hydrogen, alkyl, halogen and nitro, wherein alkyl or alkyl of said RₐR_{b}Nalkyl, RₐR_{b}Ncarboxyalkyl and arylalkyl are each members independently selected from the group consisting of C₁-alkyl, C₂-alkyl, C₃-alkyl, C₄-alkyl, C₅-alkyl, C₆-alkyl, C₇-alkyl, C₈-alkyl, C₉-alkyl or C₁₀-alkyl.

According to one embodiment of the present invention there is provided a method of treating disorders regulated by ghrelin including obesity, eating disorders, weight gain and diabetes mellitus.

According to still another embodiment, the present invention is directed to a pharmaceutical composition comprising a therapeutically effective amount of a compound of the present invention in combination with a pharmaceutically suitable carrier.

Specific compounds of formula (I) include, but are not limited to:

3-(2,6-dichlorophenyl)-N-[4-(diethylamino)phenyl]-5-methylisoxazole-4-carboxamide;

3-(2-chloro-6-fluorophenyl)-N-[4-(diethylamino)phenyl]-5-methylisoxazole-4-carboxamide;

5-but-3-enyl-3-(2,6-dichlorophenyl)-N-[4-(diethylamino)phenyl]isoxazole-4-carboxamide;

3-(2,6-dichlorophenyl)-N-[4-(diethylamino)phenyl]-5-(3,4-dihydroxybutyl)isoxazole-4-carboxamide;

3-(2,6-dichlorophenyl)-N-[4-(diethylamino)phenyl]-5-ethylisoxazole-4-carboxamide;

3-(2-chloro-6-nitrophenyl)-N-[4-(diethylamino)phenyl]-5-methylisoxazole-4-carboxamide;

3-(2,6-dichlorophenyl)-N-[4-(diethylamino)phenyl]-5-(4-hydroxybutyl)isoxazole-4-carboxamide;

3-(2,6-dichlorophenyl)-N-[4-(diethylamino)-2-methylphenyl]-5-methylisoxazole-4-carboxamide;

N-[4-(diethylamino)phenyl]-5-methyl-3-(2-nitrophenyl)isoxazole-4-carboxamide;

3-(2,6-dichlorophenyl)-N-{4-[ethyl(isopropyl)amino]phenyl} -5-methylisoxazole-4-carboxamide;

5-(4-aminobutyl)-3-(2,6-dichlorophenyl)-N-[4-(diethylamino)phenyl]isoxazole-4-carboxamide;

3-(2-bromophenyl)-N-[4-(diethylamino)phenyl]-5-methylisoxazole-4-carboxamide;

3-(2,6-dichlorophenyl)-N-[4-(diethylamino)phenyl]-5-propylisoxazole-4-carboxamide;

3-(2,6-dichlorophenyl)-N-[4-(diethylamino)phenyl] -5-isopropylisoxazole-4-carboxamide;

3-(2,6-dichlorophenyl)-N-[4-(diethylamino)phenyl]-5-(2-furyl)isoxazole-4-carboxamide;

3-(2,6-dichlorophenyl)-N-[4-(diethylamino)-2-methoxyphenyl]-5-methylisoxazole-4-carboxamide;

N-{4-[tert-butyl(ethyl)amino]phenyl}-3-(2,6-dichlorophenyl)-5-methylisoxazole-4-carboxamide;

3-(2,6-dichlorophenyl)-N-[4-(diethylamino)-2-hydroxyphenyl]-5-methylisoxazole-4-carboxamide;

N-{4-[(2-chloroethyl)(ethyl)amino]phenyl}-3-(2,6-dichlorophenyl)-5-methylisoxazole-4-carboxamide;

3-(2,6-dichlorophenyl)-N-{4-[ethyl(propyl)amino]phenyl}-5-methylisoxazole-4-carboxamide;

N-{4-[butyl(ethyl)amino]phenyl}-3-(2,6-dichlorophenyl)-5-methylisoxazole-4-carboxamide;

3-(2,6-dichlorophenyl)-N-[4-(diethylamino)-2-methylphenyl]-5-propylisoxazole-4-carboxamide;

3-(2,6-dichlorophenyl)-N-[4-(diethylamino)-2-(piperidin-1-ylcarbonyl)phenyl]-5-methylisoxazole-4-carboxamide;

3-(2,6-dichlorophenyl)-N-[4-(diethylamino)phenyl]-5-(2-phenylethyl)isoxazole-4-carboxamide;

3-(2,6-dichlorophenyl)-N-[4-(diethylamino)-2-ethylphenyl]-5-methylisoxazole-4-carboxamide;

3-(2,6-dichlorophenyl)-N-[4-(diethylamino)phenyl]-5-[3-(dimethylamino)-3-oxopropyl]isoxazole-4-carboxamide;

3-(2,6-dichlorophenyl)-N-[4-(diethylamino)phenyl]-5-[2-(1,3-dioxan-2-yl)ethyl]isoxazole-4-carboxamide;

5-[4-(acetylamino)butyl]-3-(2,6-dichlorophenyl)-N-[4-(diethylamino)phenyl]isoxazole-4-carboxamide;

3-(2,6-dichlorophenyl)-N-[4-(diethylamino)phenyl]-5-{4-[(methylsulfonyl)amino]butyl}isoxazole-4-carboxamide;

3-(2,6-dichlorophenyl)-N-[4-(diethylamino)phenyl]-5-[3-(1,3-dioxan-2-yl)propyl]isoxazole-4-carboxamide;

3-(2,6-dichlorophenyl)-N-[4-(diethylamino)phenyl]-5-(3-hydroxypropyl)isoxazole-4-carboxamide;

3-(2,6-dichlorophenyl)-N-[4-(diethylamino)phenyl]-5-(2-hydroxy-2-phenylethyl)isoxazole-4-carboxamide;

3-(2,6-dichlorophenyl)-N-[4-(diethylamino)phenyl]-5-(2-tetrahydro-2H-pyran-2-ylethyl)isoxazole-4-carboxamide;

3-(2,6-dichlorophenyl)-N-[4-(diethylamino)phenyl]-5-(tetrahydro-2H-pyran-4-ylmethyl)isoxazole-4-carboxamide;

5-butyl-3-(2,6-dichlorophenyl)-N-[4-(diethylamino)phenyl]isoxazole-4-carboxamide;

3-(2,6-dichlorophenyl)-5-[2-(1,3-dioxan-2-yl)ethyl]-N-{2-[(2-methyl-1,2,3,4-tetrahydroisoquinolin-1-yl)methyl]phenyl}isoxazole-4-carboxamide;

3-(2,6-dichlorophenyl)-N-[4-(diethylamino)phenyl]-5-[(2-oxopyrrolidin-1-yl)methyl]isoxazole-4-carboxamide;

N-(2-{[(1,3-benzodioxol-5-ylmethyl)amino]carbonyl}phenyl)-3-(2,6-dichlorophenyl)-5-methylisoxazole-4-carboxamide;

3-(2,6-dichlorophenyl)-N-[4-(diethylamino)phenyl]-5-[2-(2-oxopyrrolidin-1-yl)ethyl]isoxazole-4-carboxamide;

3-(2,6-dichlorophenyl)-N-[4-(diethylamino)phenyl]-5-[2-(3-methyl-2-oxoimidazolidin-1-yl)ethyl]isoxazole-4-carboxamide;

3-(2,6-dichlorophenyl)-N-[4-(diethylamino)phenyl]-5-[2-(dimethylamino)-2-oxoethyl]isoxazole-4-carboxamide;

3-(2,6-dichlorophenyl)-N-[4-(diethylamino)phenyl]-5-[2-(3,3-dimethyl-2-oxopyrrolidin-1-yl)ethyl]isoxazole-4-carboxamide;

3-(2,6-dichlorophenyl)-N-[4-(diethylamino)cyclohexyl]-5-methylisoxazole-4-carboxamide;

3-(2,6-dichlorophenyl)-N-[4-(diethylamino)-2-methylphenyl]-5-[2-(1,3-dioxan-2-yl)ethyl]isoxazole-4-carboxamide;

3-(2,6-dichlorophenyl)-N-[4-(diethylamino)phenyl]-5-[2-(1,3-dioxolan-2-yl)ethyl]isoxazole-4-carboxamide;

3-(2,6-dichlorophenyl)-N-[4-(diethylamino)phenyl]-5-(2-methoxyethyl)isoxazole-4-carboxamide;

3-(2,6-dichlorophenyl)-N-[4-(diethylamino)phenyl]-5-(2-tetrahydrofuran-2-ylethyl)isoxazole-4-carboxamide;

3-(2,6-dichlorophenyl)-N-[2-(3,4-dihydroisoquinolin-2(1H)-ylcarbonyl)phenyl]-5-methylisoxazole-4-carboxamide;

3-(2,6-dichlorophenyl)-N-(2-{[(2,3-dihydro-1-benzofuran-5-ylmethyl)amino]carbonyl}phenyl)-5-methylisoxazole-4-carboxamide;

3-(2,6-dichlorophenyl)-N-{(1R)-1-[4-(diethylamino)phenyl]ethyl}-5-methylisoxazole-4-carboxamide;

3-(2,6-dichlorophenyl)-N-[4-(diethylamino)phenyl]-5-[2-(2-oxopiperidin-1-yl)ethyl]isoxazole-4-carboxamide;

5-{2-[acetyl(methyl)amino]ethyl}-3-(2,6-dichlorophenyl)-N-[4-(diethylamino)phenyl]isoxazole-4-carboxamide;

3-(2,6-dichlorophenyl)-N-(4-{2-[(3-ethoxypropyl)amino]ethyl}phenyl)-5-methylisoxazole-4-carboxamide;

3-(2,6-dichlorophenyl)-N-(4-{2-[(3-isopropoxypropyl)amino]ethyl}phenyl)-5-methylisoxazole-4-carboxamide; and

3-(2,6-dichlorophenyl)-5-methyl-N-(4-{2-[(2-phenoxyethyl)amino]ethyl}phenyl)isoxazole-4-carboxamide.

The present compounds may exist as therapeutically suitable salts. The term “therapeutically suitable salt,” refers to salts or zwitterions of the compounds which are water or oil-soluble or dispersible, suitable for treatment of disorders without undue toxicity, irritation, and allergic response, commensurate with a reasonable benefit/risk ratio, and effective for their intended use. The salts may be prepared during the final isolation and purification of the compounds or separately by reacting an amino group of the compounds with a suitable acid. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetic, trifluoroacetic, glutamate, para-toluenesulfonate, undecanoate, hydrochloric, hydrobromic, sulfuric, phosphoric, and the like. The amino groups of the compounds may also be quaternized with alkyl chlorides, bromides, and iodides such as methyl, ethyl, propyl, isopropyl, butyl, lauryl, myristyl, stearyl, and the like.

Basic addition salts may be prepared during the final isolation and purification of the present compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts derived from methylamine, dimethylamine, trimethylamine, triethylamine, diethylamine, ethylamine, tributlyamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine, ethylenediamine, ethanolamine, diethanolamine, piperidine, piperazine, and the like, are contemplated as being within the scope of the present invention.

The present compounds may also exist as therapeutically suitable prodrugs. The term “therapeutically suitable prodrug,” refers to those prodrugs or zwitterions which are suitable for use in contact with the tissues of patients without undue toxicity, irritation, and allergic response, are commensurate with a reasonable benefit/risk ratio, and are effective for their intended use. The term “prodrug,” refers to compounds which are rapidly transformed in vivo to the parent compounds of the present invention for example, by hydrolysis in blood.

Asymmetric centers may exist in the present compounds. Individual stereoisomers of the compounds are prepared by synthesis from chiral starting materials or by preparation of racemic mixtures and separation by conversion to a mixture of diastereomers followed by separation or recrystallization, chromatographic techniques, or direct separation of the enantiomers on chiral chromatographic columns. Starting materials of particular stereochemistry are either commercially available or are made by the methods described hereinbelow and resolved by techniques well-known in the art.

Geometric isomers may exist in the present compounds. The invention contemplates the various geometric isomers and mixtures thereof resulting from the disposal of substituents around a carbon-carbon double bond, a cycloalkyl group, or a heterocycloalkyl group. Substituents around a carbon-carbon double bond are designated as being of Z or E configuration and substituents around a cycloalkyl or heterocycloalkyl are designated as being of cis or trans configuration.

Therapeutic compositions of the present compounds comprise an effective amount of the same formulated with one or more therapeutically suitable excipients. The term “therapeutically suitable excipient,” as used herein, represents a non-toxic, solid, semi-solid or liquid filler, diluent, encapsulating material, or formulation auxiliary of any type. Examples of therapeutically suitable excipients include sugars; cellulose and derivatives thereof; oils; glycols; solutions; buffering, coloring, releasing, coating, sweetening, flavoring, and perfuming agents; and the like. These therapeutic compositions may be administered parenterally, intracisternally, orally, rectally, or intraperitoneally.

Liquid dosage forms for oral administration of the present compounds comprise formulations of the same as emulsions, microemulsions, solutions, suspensions, syrups, and elixirs. In addition to the compounds, the liquid dosage forms may contain diluents and/or solubilizing or emulsifying agents. Besides inert diluents, the oral compositions may include wetting, emulsifying, sweetening, flavoring, and perfuming agents. Injectable preparations of the present compounds comprise sterile, injectable, aqueous and oleaginous solutions, suspensions or emulsions, any of which may be optionally formulated with parenterally suitable diluents, dispersing, wetting, or suspending agents. These injectable preparations may be sterilized by filtration through a bacterial-retaining filter or formulated with sterilizing agents which dissolve or disperse in the injectable media.

Regulation of the effects of ghrelin by the compounds of the present invention may be delayed by using a liquid suspension of crystalline or amorphous material with poor water solubility. The rate of absorption of the compounds depends upon their rate of dissolution which, in turn, depends on their crystalline form. Delayed absorption of a parenterally administered compound may be accomplished by dissolving or suspending the compound in oil. Injectable depot forms of the compounds may also be prepared by microencapsulating the same in biodegradable polymers. Depending upon the ratio of compound to polymer and the nature of the polymer employed, the rate of release may be controlled. Depot injectable formulations are also prepared by entrapping the compounds in liposomes or microemulsions which are compatible with body tissues.

Solid dosage forms for oral administration of the present compounds include capsules, tablets, pills, powders, and granules. In such forms, the compound is mixed with at least one inert, therapeutically suitable excipient such as a carrier, filler, extender, disintegrating agent, solution retarding agent, wetting agent, absorbent, or lubricant. With capsules, tablets, and pills, the excipient may also contain buffering agents. Suppositories for rectal administration may be prepared by mixing the compounds with a suitable non-irritating excipient which is solid at ordinary temperature but fluid in the rectum.

The present compounds may be micro-encapsulated with one or more of the excipients discussed previously. The solid dosage forms of tablets, dragees, capsules, pills, and granules may be prepared with coatings and shells such as enteric and release-controlling. In these forms, the compounds may be mixed with at least one inert diluent and may optionally comprise tableting lubricants and aids. Capsules may also optionally contain opacifying agents which delay release of the compounds in a desired part of the intestinal tract.

Transdermal patches have the added advantage of providing controlled delivery of the present compounds to the body. Such dosage forms are prepared by dissolving or dispensing the compounds in the proper medium. Absorption enhancers may also be used to increase the flux of the compounds across the skin, and the rate of absorption may be controlled by providing a rate controlling membrane or by dispersing the compounds in a polymer matrix or gel.

Disorders that may be regulated by ghrelin are treated or prevented in a mammal by administering to the mammal, a therapeutically effective amount of compound of the present invention in such an amount and for such time as is necessary to achieve the desired result. The term “therapeutically effective amount,” refers to a sufficient amount of a compound to effectively emeliorate disorders reglulated by ghrelin at a reasonable benefit/risk ratio applicable to any medical treatment. The specific therapeutically effective dose level for any particular mammal will depend upon a variety of factors including the disorder being treated and the severity of the disorder; the activity of the compound employed; the specific composition employed; the age, body weight, general health, sex, and diet of the mammal; the time of administration, route of administration, rate of excretion; the duration of the treatment; and drugs used in combination or coincidental therapy.

The total daily dose of the present compounds in single or divided doses may be in amounts, for example, from 0.01 to 50 mg/kg body weight or more usually from 0.1 to 25 mg/kg body weight. In general, treatment regimens comprise administration to a mammal in need of such treatment from about 10 mg to about 1000 mg of the compounds per day in single or multiple doses.

### Determination of Biological Activity

The activities of the ghrelin receptor modulators, including both agonists and antagonists, have been determined using a primary binding assay and a secondary functional assay.

Primary Radiolabelled Ligand Competetition Binding Assay

Ghrelin binding assays were performed with membrane preparations. CHO-K cells expressing human ghrelin receptor (Euroscreen) were suspended in sucrose buffer (0.25 M sucrose, 10 mM hepes pH 7.4, 1 mM PMSF, 5 μg/ml pepstain-A, 3 mM EDTA and 0.025% bacitracin) and disrupted by sonication using a vibra cell (Sonics and Materials Inc.) on 70% duty cycle in 15-second pulses on ice for 2.5 min. The homogenate was centrifuiged at 60,000×g for 60 minutes and pellets were suspended in tris buffer (20 mM tris pH 7.4, 5 ug/ml pepstatin-A, 0.1 mM PMSF and 3mM EDTA). Binding reactions contained 1 ug membrane as determined by BCA protein assay (Pierce), 0.1 nM [¹²⁵I]-ghrelin (PerkinElmer) with or without compound addition in 100 ul of binding buffer (25 mM Hepes pH 7.4, 1 mM CaCl₂, 5 mM MgSO₄ and 0.5% protease free BSA). Incubations were carried out at room temperature for 2 hr and were terminated by filtration using Filtermate Harvester (PerkinElmer) onto GF/C filter plates (Millipore) previously soaked in 0.5% polyethylenimine for 2 hours. Bound [¹²⁵I]-ghrelin was determined by scintillation counting using Top Count NXT (PerkinElmer). The effects of compound were expressed as % inhibiton of [¹²⁵I]-ghrelin binding. Sigmoidal curves were fitted by Assay Explorer (MDL) software and IC₅₀ values determined.

Secondary Fluorescent Calcium Indicator Assay (FLIPR)

CHO-K cells expressing human GHS receptor (Euroscreen) were plated in black 96-well plates with clear bottom (Costar) and cultured to confluency overnight in growth media (Ultra-CHO from BioWhittaker supplemented with 1% dialyzed FCS, 1% penicillin/streptomycin/fungizone, and 400 ug/ml G418 all from Life Technologies) at 37° C. in a humidified cell incubator containing 5% CO₂. Growth media was aspirated and replaced with 100 ul of Dulbecco's phosphate-buffered saline (DPBS) containing 1,000 mg/l D-glucose, 36 mg/l sodium pyruvate, without phenol red (Life Technologies) with 1.14 mM Fluo-4 AM (Molecular Probes) and 0.25 M probenecid (Sigma) for 1 to 3 hours in the dark at room temperature. The dye solution was aspirated and the cells were washed twice with DPBS using the EL-450X cell washer (BioTech). After the last wash, 100 ul of DPBS was added to each well. Cell plates were then transferred to the FLIPR unit (Molecular Probes). Compound additions were 50 ul in duplicate of 4× final concentration in DPBS containing 0.1% BSA and 4% DMSO. Fluorescence emissions from 96 wells were measured simultaneously at excitation and emission wavelength of 488 and 520 mn, respectively for 3 minutes in 1-second intervals for the first minute and 5-second intervals thereafter. During this time agonist responses, if any, were recorded in the absence of ghrelin. Next, 50 ul in duplicate of 4× concentrated ghrelin (Bachem) solution in DPBS containing 0.1% BSA and 4% DMSO were delivered within 1 second by an integrated 96-well pipettor to a final concentration of 1 nM. Fluorescence emissions were measured for another 3 minutes as above. During this time the antagonist effects of compounds on ghrelin-stimulated calcium flux were recorded and expressed as % inhibition of the maximal ghrelin response (10 nM). Sigmoidal curves were fitted by Assay Explorer (MDL) software and IC₅₀ values determined. In addition, the agonist effects of the compounds could also be obtained and expressed as % maximal ghrelin response (10 nM). Sigmoidal curves were fitted by Assay Explorer (MDL) software and EC₅₀ values determined.

The instant compounds were found to modulate the activity of the ghrelin receptor with IC₅₀/EC₅₀ in a range of about 0.001 μM to about 10 μM in both the binding and FLIPR assays. In a preferred range, the compounds modulated the ghrelin receptor with IC₅₀/EC₅₀ in a range of about 0.001 μM to about 1.0 μM in both the binding and FLIPR assays; and in a more preferred range, the compounds modulated the ghrelin receptor with IC₅₀/EC₅₀ in a range of about 0.001 μM to about 0.2 μM in both the binding and FLIPR assays.

Synthetic Methods

Abbreviations which have been used in the descriptions of the scheme and the examples that follow are: BBr₃ for boron tribromide; m-CPBA for meta-chloroperoxy-benzoic acid; DMF for N,N-dimethylformamide; DMSO for dimethylsulfoxide; DEAD for diethyl azodicarboxylate; EDAC for 1-(3-dimethylaminopropyl)-3-ethylcarbodiimide hydrochloride; HATU for O-(7-azabenzotriazol-1-yl)-N,N,N′N′-tetramethyluronium hexafluorophosphate; HOBT for 1-hydroxybenzotriazole hydrate; NMP for N-methylpyrrolidinone; NCS for N-chlorosuccinimide; MeONa for sodium methoxide; MeOH for methanol; MTBE for methyl tert butyl ether; THF for tetrahydrofuran; TFA for trifluoroacetic acid; TBAF for tetra butylammonium fluoride; Pd(dppf)Cl₂ for (diphenylphospino)ferrocenyl palladium chloride; Ph₃P for triphenylphosphine; Pr₂Net for diisopropyl ethylamine; and TBTU for (benzotriazol-1-yloxy)-dimethylamino-methylene)-dimethyl-ammonium tetrafluoroborate.

Schemes

The compounds and processes of the present invention will be better understood in connection with the following synthetic schemes which illustrate the methods by which the compounds of the invention may be prepared. The groups R₁, R₂, R₃ and R₄ are as defined above unless otherwise noted below.

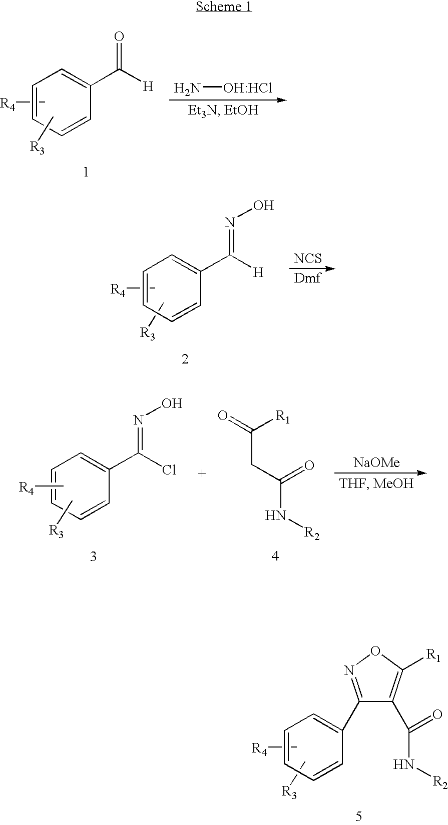

As shown in Scheme 1, compounds of formula 1 (wherein R₃ and R₄ are defined within the scope of this document) may be treated with hydroxylamine hydrochloride in the presence of a base such as but not limited to triethylamine in solvents such as but not limited to ethanol, pyridine, or THF and the like to form compounds of formula 2. Compounds of formula 2 may be treated with N-chlorosuccinimide in solvents such as but not limited to DMF to provide compounds of formula 3. Compounds of formula 3 may be treated with compounds of formula 4 in the presence of sodium methoxide in solvent such as but not limited to THF to provide compounds of formula 5 which are representative of compounds the present invention.

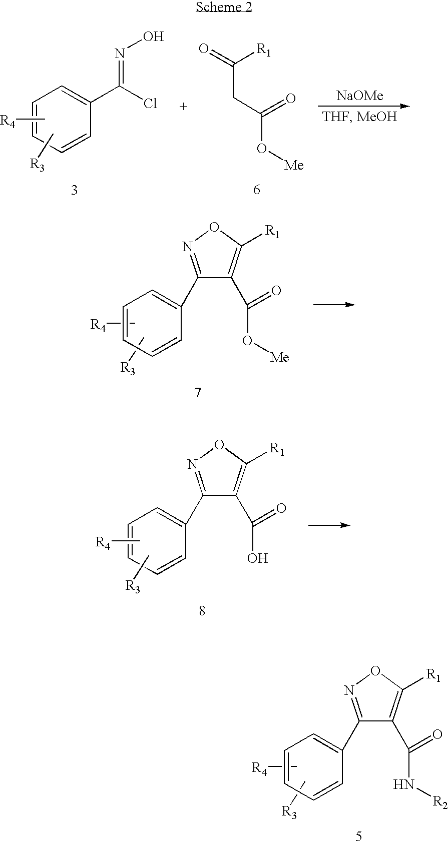

Alternatively, as shown in Scheme 2, when compounds of formula 3 are treated with beta-keto esters of formula 6 using the same conditions described in Scheme 1, compounds of formula 7 may be obtained. The hydrolysis of the ester group using conditions known to those skilled in the art including but not limited to lithium hydroxide, sodium hydroxide and lithium hydroxide in solvents such as but not limited to aqueous methanol, aqueous THF and aqueous dioxane may be used to obtain compounds of formula 8. Compounds of formula 8 may be treated using condition known to those skilled in the art to generate amides from amines and carboxylic acids. Common conditions known to those skilled in the art that may be used in the transformation of carboxylic acid of the present invention to the corresponding amide of the present inventions include but are not limited to stirring the carboxylic acid and one of (benzotriazol-1-yloxy)-dimethylamino-methylene)-dimethyl-ammonium tetrafluoroborate (TBTU), N-hydroxybenzotriazole (HOBT) and triethylamine in DMF; (ethyl dimethylpropylcarbodiimide:hydrochloride (EDCI), HOBT and N-methylmorpholine in dichloromethane followed by the addition of the amine and a base such as N-methyl morpholine to provide the compound of formula 5. Alternatively, pretreatment of the acid with thionyl chloride in refluxing dichlormethane followed by the addition of the amine and a base such as N-methyl morpholine may also be used to generate compounds of formula 5.

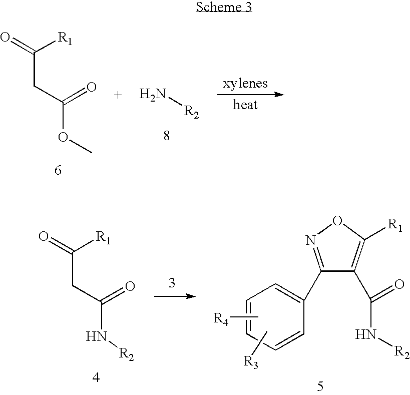

Alternatively, beta-keto esters of formula 6 may be treated with amines of formula 8 under heated conditions to produce beta-keto amides of formula 4. Compounds of formula 4 may be treated with compounds of formula 3 as described in Scheme 1 to produce compounds of formula 5 which are representative of compounds of formula (I). This alternative Scheme may be utilized as another method to generate compounds of formula (I) when an alternative synthetic method is needed based on availability of starting materials or difficulties in obtaining products as determined by those skilled in the art.

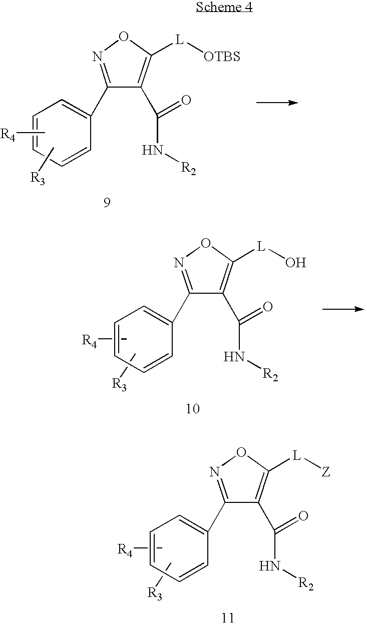

Compounds of formula 9, wherein R₁ consist of a tert-butyldimethylsilyl ether attached to the parent molecule through a linker L which may be a member selected from the group consisting of alkyl and alkenyl, may be made through the treatment of compounds of formula 4 which contains the silyl ether Linker with compounds of formula 3 according to the methods outlined in any one of the Schemes 1-3. The silyl ether group may be removed using tetra-butyl ammonium fluoride in THF or through methods known to those skilled in the art or as described in Greenes “Protecting groups in Organic Chemistry” 3rd ed. (1999, Wiley & Sons, Inc.) to provide compounds of formula 10. The alcohol portion of compounds of formula 10 may be further treated using methods known to those skilled in the art to incorporated other functional groups such as aryl, heterocycle and RₐR_{b}N—. Representative methods useful for such transformation include but are not limited to variations of the Mitsunobu reactions as described in “Advanced Organic Chemistry” 3^{rd} ed. March (1985, Wiley & Sons, Inc.). Other methods include the treatment of the alcohol with methanesulfonyl chloride and triethylamine in dichloromethane to provide the mesylate. The mesylate functional group is known to those skilled in the art to be converted to other functional groups when treated with various nucleophilic reagents. Such methods allow for the incorporation of such groups that include but are not limited to heterocycle groups. Such methods are considered to be within the scope of the present invention and are thus incorporated herein by reference.

The present invention will now be described in connection with certain embodiments which are not intended to limit its scope. On the contrary, the present invention covers all alternatives, modifications, and equivalents as may be included within the scope of the claims. Thus, the following examples, which include preferred embodiments, will illustrate the preferred practice of the present invention, it being understood that the examples are for the purposes of illustration of certain preferred embodiments.

Compounds of the invention were named by ACD/ChemSketch version 5.01 (developed by Advanced Chemistry Development, Inc., Toronto, ON, Canada) or were given names which appeared to be consistent with ACD nomenclature.

### Experimentals

### EXAMPLE 1

### 3-(2,6-dichlorophenyl)-N-[4-(diethylamino)phenyl]-5-methylisoxazole-4-carboxamide

To 3-(2,6-dichloro-phenyl)-5-methyl-isoxazole-4-carboxylic acid (2.2 g, 8.0 mmol) in DMF (10 mL) was added N,N-diethylphenylene diamine (1.4 g, 8.0 mmol), Et₃N (0.81 g, 8.0 mmol), and TBTU (2.6 g, 8.0 mmol) at 0° C. After 2 hours, ice chips and 100 mL ethyl acetate were added. Organic layer was washed with water (3×30 mL), dried over MgSO₄, and then concentrated under reduced pressure. Purification by column chromatography provided the titled compound (1.5 g, 45%). ¹H NMR (300 MHz, CDCl₃) δ 7.53 (d, J =9.3 Hz, 1H), 7.53 (d, J=6.9 Hz, 1H), 7.46 (dd, J=6.3, 9.3 Hz, 1H), 7.03 (d, J=8.7 Hz, 2H), 6.69 (br s, 1H), 6.56 (d, J=9.0Hz, 2H), 3.29 (q, J=7.2 Hz, 4H), 2.85 (s, 3H), and 1.1 (t, J=7.2 Hz, 6H); MS (ESI) positive ion 418(M+H)⁺, 420 (M+H)⁺; negative ion 416 (M−H)⁻, 418 (M−H)⁻.

### EXAMPLE 2

### 3-(2-chloro-6-fluorophenyl)-N-[4-(diethylamino)phenyl]-5-methylisoxazole-4-carboxamide

The titled compound was prepared according to the procedure described in Example 1, substituting 3-(2-chloro-6-fluorophenyl)-5-methyl-isoxazole-4-carboxylic acid for 3-(2,6-dichlorophenyl)-5-methyl-isoxazole-4-carboxylic acid. ¹H NMR (300 MHz, DMSO-d₆) δ 9.82 (s, 1H), 7.63-7.34 (m, 3H), 7.31 (d, J=9.2 Hz, 2H), 6.60 (d, J=9.2 Hz, 2H), 3.28 (q, J=7.1 Hz, 4H), 2.69 (s, 3H), 1.05 (t, J=7.1 Hz, 6H); MS (ESI(+)) m/e 402 (M+H)⁺.

### EXAMPLE 3

### 5-But-3-enyl-3-(2,6-dichlorophenyl)-N-[4-(diethylamino)phenyl]isoxazole-4-carboxamide

### EXAMPLE 3A

### 5-But-3-enyl-3-(2,6-dichloro-phenyl)-isoxazole-4-carboxylic acid

To a stirred solution of 3-(2,6-dichloro-phenyl)-5-methyl-isoxazole-4-carboxylic acid (250 mg, 0.92 mmol) in THF at −78° C. under N₂ was added n-BuLi (2.5 M, 0.77 mL, 1.93 mmol) dropwise. The resulting yellow slurry was stirred at −78 ° C. for 2 hours after which allyl iodide was added. The mixture was allowed to warm to room temperature slowly over a period of 2 hours and stirred at room temperature for one hour. Aqueous NaOH (3 N, 1.3 mL) was added and stirred for one hour at room temperature. The reaction mixture was then acidified to pH ˜3 with 3 N HCl, and extracted with dichloromethane (2×15 mL). The combined organic layer was washed with brine, dried over Na₂SO₄, filtered and concentration under reduced pressure to provide the titled compound (180 mg, 63%). MS (ESI) m/e 310, 312, 314 (M−H)⁻.

### EXAMPLE 3B

### 5-But-3-enyl-3-(2,6-dichlorophenyl)-N-r4-(diethylamino)phenyl]isoxazole-4-carboxamide

The titled compound was prepared according to the procedure described in Example 1, substituting 5-but-3-enyl-3-(2,6-dichloro-phenyl)-isoxazole-4-carboxylic acid for 3-(2,6-dichlorophenyl)-5-methyl-isoxazole-4-carboxylic acid used in Example 1. ¹H NMR (300 MHz, CDCl₃) δ 7.53 (d, J=9.2 Hz, 1H), 7.53 (d, J=6.4 Hz, 1H), 7.45 (dd, J=6.3, 9.3 Hz, 1H), 7.01 (d, J=9.0 Hz, 2H), 6.73 (bs, 1H), 6.56 (d, J=9.0 Hz, 2H), 5.99-5.72 (m, 1H), 5.10 (dd, J=17.3, 1.7 Hz, 1H), 5.05 (d, J=10.2 Hz, 1H), 3.36 (t, J=7.4 Hz, 2H), 3.29 (q, J=6.8 Hz, 4H), 2.62 (q, J=7.4 Hz, 2H), 1.10 (t, J=6.8 Hz, 6H). MS (ESI) m/e 458, 460, 462 (M+H)⁺.

### EXAMPLE 4

### 3-(2,6-dichlorophenyl)-N-[4-(diethylamino)phenyl]-5-(3,4-dihydroxybutyl)isoxazole-4-carboxamide

The olefin (145 mg, 0.32 mmol) from Example 3B was dissolved in 3 mL of a mixture of acetone/H₂O (5:1, v/v) at room temperature. N-Methyl morpholine N-oxide (45 mg, 0.38 mmol) was added, followed by a catalytic amount of OsO₄ in n-BuOH. The resulting mixture was then stirred at room temperature for 16 hours. The reaction mixture was then diluted with aqueous Na₂S₂O₃, extracted with ethyl acetate, (2×15 mL). The combined organic layer was washed with brine, dried (Na₂SO₄), filtered and concentrated under reduced pressure. The residue was purified on a Gilson preparative HPLC to provide the titled compound (120 mg, 0.24 mmol, 75% yield). ¹H NMR (300 MHz, CDCl₃) δ 9.19 (s, 1H), 7.62 (d, J=9.2 Hz, 1H), 7.62 (d, J=6.4 Hz, 1H), 7.51 (dd, J=6.3, 2.7 Hz, 1H), 7.44 (bs, 3H), 7.42 (dd, J=6.3, 0.9 Hz, 1H), 3.82-3.63 (m, 2H), 3.63-3.38 (m, 6H), 3.21 (td, J=15.0, 5.0 Hz, 1H), 2.20-1.95 (m, 2H), 1.11 (t, J=7.0 Hz, 6H). MS (ESI) m/e 492, 494, 496 (M+H)⁺.

### EXAMPLE 5

### 3-(2,6-dichlorophenyl)-N-[4-(diethylamino)phenyl]-5-ethylisoxazole-4-carboxamide

The titled compound was prepared according to the procedure described in Example 1, substituting 3-(2,6-dichlorophenyl)-5-ethyl-isoxazole-4-carboxylic acid for 3-(2,6-dichlorophenyl)-5-methyl-isoxazole-4-carboxylic acid. MS (ESI(+)) m/e 432 (M+H)⁺; ¹H NMR (300 MHz, DMSO-d₆) δ 9.71 (s, 1H), 7.61-7.50 (m, 3H), 7.27 (d, J=8.9 Hz, 2H), 6.58 (d, J=9.2 Hz, 2H), 3.27 (q, J=7.1 Hz, 4H), 3.12 (q, J=7.7 Hz, 2H), 1.32 (t, J=7.7 Hz, 3H), 1.03 (t, J=7.1 Hz, 6H).

### EXAMPLE 6

### 3-(2-chloro-6-nitrophenyl)-N-[4-(diethylamino)phenyl]-5-methylisoxazole-4-carboxamide

A mixture of 2-chloro-6-nitro-benzaldehye (530 mg, 2.9 mmol), hydroxylamine hydrochloride (209 mg, 3.0 mmol), and Et₃N (303 mg, 3.0 mmol) in ethanol (4 mL) was heated at 75° C. for 16 hours. The solvent was removed under reduced pressure to provide the crude oxime (500 mg). This material was dissolved in DMF (5 mL), to which N-chlorosuccinimide (300 mg, 2.5 mmol) was added after which the mixture was allowed to stir at room temperature for 4 hours. Ethyl acetate (100 mL) was added and the mixture was washed with water (2×50 mL). The organic extracts were dried over MgSO₄, filtered and concentrated under reduced pressure to provide chlorooximidate (700 mg). This material was dissolved in THF (3 mL) and a solution of N-(4-diethylamino-phenyl)-3-oxo-butyramide in THF (3 mL) was added followed by addition of MeONa (0.5 M in MeOH, 10 mL, 5 mmol) at 0° C. The reaction mixture was quenched with aqueous NH₄Cl (Sat'd) solution (10 mL) after 16 hour and extracted with ethyl acetate (3×20 mL). The combined organic extracts were dried over MgSO₄, filtered and concentrated under reduced pressure. Column chromatography (hexanes:ethyl acetate=2:1) purification provided the titled compound (530 mg, 43% over 3 steps). ¹H NMR (300 MHz, DMSO-d₆) δ 9.76 (s, 1H), 8.19 (d, J=8.1 Hz, 1H), 8.02 (d, J=8.1 Hz, 1H), 7.81 (t, J=8.7 Hz, 1H), 7.29 (d, J=9.3 Hz, 2H), 6.58 (d, J=9.0 Hz, 2H), 3.27 (q, J=7.8 Hz, 4H), 2.73 (s, 3H), and 1.04 (t, J=7.2 Hz, 6H). MS (ESI) positive ion 429 (M+H)⁺; negative ion 427 (M−H)⁻.

### EXAMPLE 7

### 3-(2,6-dichlorophenyl)-N-[4-(diethylamino)phenyl]-5-(4-hydroxybutyl)isoxazole-4-carboxamide

### EXAMPLE 7A

### 5-[4-(tert-butyl-dimethyl-silanyloxy)butyl]-3-(2,6-dichloro-phenyl)-isoxazole-4-carboxylic acid-(4-diethylamino-phenyl)-amide

The titled compound was prepared according to the procedure described in Example 3, substituting tert-butyl-(3-iodo-propoxy)-dimethyl-silane for allyl iodide used in Example 3A.

### EXAMPLE 7B

### 3-(2,6-dichlorophenyl)-N-[4-(diethylamino)phenyl]-5-(4-hydroxybutyl)isoxazole-4-carboxamide

To the silyl ether (408 mg, 0.69 mmol) from Example 7A in THF was added solid TBAF (271 mg, 1.04 mmol). The reaction mixture was stirred at room temperature for 2 h before it was poured into 1 N HCl and extracted with Ethyl acetate (20 mL). The organic layer was washed with brine, dried over Na₂SO₄, and evaporated in vacuo. The crude product was purified on an Alltech Sep-Pak, eluting with 20% ethyl acetate/Hexanes to provide the titled alcohol as a light brown oil (250 mg, 45% over two steps). ¹H NMR (300 MHz, CDCl₃) δ 7.54 (dd, J=9.3 Hz, 1H), 7.52 (d, J=6.3 Hz, 1H), 7.46 (dd, J=9.3, 6.3 Hz, 1H), 7.01 (q, J=9.0 Hz, 2H), 6.78 (bs, 1H), 6.55 (d, J=9.0 Hz, 2H), 3.74 (q, J=7.8 Hz, 2H), 3.29 (q, J=7.0 Hz, 4H), 1.99 (pentet, J=6.1 Hz, 2H), 1.73 (heptet, J=6.1 Hz, 2H), 1.60 (s, 1H), 1.11 (t, J=7.0 Hz, 6H). MS (ESI) m/e 476, 478, 4.80 (M+H)⁺.

### EXAMPLE 8

### 3-(2,6-dichlorophenyl)-N-[4-(diethylamino)-2-methylphenyl]-5-methylisoxazole-4-carboxamide

The titled compound was prepared according to the procedure described in Example 1, substituting N,N-diethyl-2-methyl-1,4-phenylenediamine for N,N-diethyl-1,4-phenylenediamine. ¹H NMR (300 MHz, DMSO-d₆) δ 9.19 (s, 1H), 7.63-7.50 (m, 3H), 6.97 (d, J=8.1 Hz, 1H), 6.51-6.42 (m, 2H), 3.29 (q, J=7.1 Hz, 4H), 2.77 (s, 3H), 2.06 (s, 3H), 1.05 (t, J=7.1 Hz, 6H); MS (ESI(+)) m/e 432 (M+H)⁺.

### EXAMPLE 9

### N-[4-(diethylamino)phenyl]-5-methyl-3-(2-nitrophenyl)isoxazole-4-carboxamide

### EXAMPLE 9A

### N-(4-Diethylamino-phenyl)-3-oxo-butyramide

N,N-diethyl-1,4-phenylenediamine (2.04 g, 12.2 mmol) and methyl-3-oxo-butanoate (2.82 g, 24.4 mmol) were added to xylenes (8 mL) and heated to 145° C. for 4 hours. The crude mixture was purified by column chromatography (30-60% Ethyl acetate in hexanes) to provide the titled compound 9A (1.36 g, 45%).

### EXAMPLE 9B

### Chloroximidate

2-Nitrobenzaldoxime (267 mg, 1.61 mmol) was dissolved in DMF (3 mL). NCS (215 mg, 1.61 mmol) was added and the mixture was stirred in an ambient temperature water bath for 14 hours. The reaction mixture was poured into water (50 mL) and extracted with MTBE (50 mL). The ether layer was washed with brine (50 mL×2). The titled compound 9B (318 mg, 98.8%) was obtained as a light yellow oil following solvent removal.

### EXAMPLE 9C

### N-[4-(diethylamino)phenyl]-5-methyl-3-(2-nitrophenyl)isoxazole-4-carboxamide

Compound 9A (400 mg, 1.61 mmol) was dissolved in THF (1.5 mL) followed by addition of NaOMe (3.2 mL, 0.5 M in MeOH). The reaction mixture was stirred for 20 minutes. Compound 9B (318 mg, 1.61 mmol) was added and stirred overnight. The solvent was removed and the titled compound 9C (256 mg, 40.4%) was purified by column chromatography (0-45% Ethyl acetate in hexanes). ¹H NMR (300 MHz, DMSO-d₆) δ 1.04 (t, J=6.95 Hz, 6 H), 2.67 (s, 3 H), 3.28 (q, J=7.46 Hz, 4 H), 6.59 (d, J=9.16 Hz, 2H), 7.26 (d, J=8.82 Hz, 2 H), 7.68 (d, J=7.46 Hz, 1 H), 7.79 (td, J=7.80, 1.70 Hz, 1 H), 7.87 (td, J=7.54, 1.19 Hz, 1 H), 8.17 (dd, J=7.97, 1.19 Hz, 1 H), 9.74 (s, 1 H); MS (APCI) positive ion; m/z 395.8 (M+H)⁺, 348.2 (M-NO₂)⁺.

### EXAMPLE 10

### 3-(2,6-dichlorophenyl)-N-{4-[ethyl(isopropyl)amino]phenyl}-5-methylisoxazole-4-carboxamide

This title compound was prepared according to the procedure described in Example 1, substituting the N,N-diethylphenylene diamine from Example 1 for N-ethyl-N-isopropyl-benzene-1,4-diamine hydrochloride. ¹H NMR (300 MHz, DMSO-d₆) δ 9.79 (s, 1H), 7.60 (d, J=9.6 Hz, 1H), 7.60 (d, J=7.2 Hz, 1H), 7.53 (dd, J=6.6, 9.9 Hz, 1H), 7.32 (d, K=9.0 Hz, 2H), 6.66 (d, J=9.0 Hz, 2H), 3.94 (septet, J=6.3 Hz, 1H), 3.16 (q, J=7.2 Hz, 2H), 2.71 (s, 3H), 1.10 (d, J=6.3 Hz, 6H, and 1.04 (t, J=6.6 Hz, 3H).

### EXAMPLE 11

### 5-(4-aminobutyl)-3-(2,6-dichlorophenyl)-N-[4-(diethylamino)phenyl]isoxazole-4-carboxamide

To a stirred mixture of alcohol from Example 7B (197 mg, 0.41 mmol), triphenyl phosphine (141 mg, 0.54 mmol) in anhydrous THF under N₂ was added diphenylphosphoryl azide (107 μL, 0.49 mmol) and DEAD (79 μL, 0.49 mmol) sequentially. The resulting mixture was then stirred for 1 hour before the solvent was removed and the desired azide (150 mg, 73% yield) was isolated using silica gel Sep-Pak eluting with 10-20% ethyl acetate/hexanes. To a stirred solution of azide (150 mg, 0.30 mmol) in 3.0 mL of THF was added Ph₃P (118 mg, 0.45 mmol) and 0.5 mL of water, and stirred at room temperature for 3 hours. Solvent was then removed under reduced pressure and the titled compound (45 mg, 32%) was isolated on a Gilson preparative HPLC as a light brown oil. ¹H NMR (300 MHz, DMSO-d6) δ 7.62 (d, J=2.7 Hz, 1H), 7.60-7.55 (m, 4H), 7.56 (d, J=6.1 Hz, 1H), 7.52 (d, J=6.1 Hz, 1H), 5.84 (br s, 1H), 3.43 (br m, 4H), 3.13 (t, J=7.3 Hz, 2H), 2.99 (t, J=6.4 Hz, 2H), 1.72 (pentet, J=7.3 Hz, 2H), 1.40 (pentet, J=7.3 Hz, 2H), 1.00 (t, J=7.1 Hz, 6H), MS (ESI) m/e 475, 477, 479 (M+H)⁺.

### EXAMPLE 12

### 3-(2-bromophenyl)-N-[4-(diethylamino)phenyl]-5-methylisoxazole-4-carboxamide

The titled compound was prepared according to the procedure described in Example 1, substituting 3-(2-bromo)-5-methyl-isoxazole-4-carboxylic acid for 3-(2,6-dichlorophenyl)-5-methyl-isoxazole-4-carboxylic acid. White solid; ¹H NMR (300 MHz, CDCl₃) δ 7.79 (d, J=8.1 Hz, 1H), 7.57-7.51 (m, 2H), 7.50-7.41 (m, 1H), 7.00 (d, J=9.0 Hz, 2H), 6.71 (br s, 1H), 6.56 (d, J=9.0 Hz, 2H), 3.29 (q, J=7.0 Hz, 4H), 2.83 (s, 3H), 1.11 (t, J=7.0 Hz, 6H). MS (ESI) m/e 428, 430, 432 (M+H)⁺.

### EXAMPLE 13

### 3-(2,6-dichlorophenyl)-N-[4-(diethylamino)phenyl]-5-propylisoxazole-4-carboxamide

The titled compound was prepared according to the procedure described in Example 14, substituting 3-oxo-hexanoic acid methyl ester for methyl isobutyrylacetate used in Example 14A. ¹H NMR (300 MHz, DMSO-d6) δ 0.92 (t, J=7.46 Hz, 3 H), 1.04 (t, J=6.78 Hz, 6 H), 1.75 (sextet, J=7.46 Hz, 2 H), 3.09 (t, J=7.29 Hz, 2 H), 3.28 (q, J=6.78 Hz, 4 H), 6.59 (d, J=9.15 Hz, 2 H), 7.28 (d, J=8.81 Hz, 2 H), 7.53 (dd, J=9.49, 6.10 Hz, 1 H), 7.60 (d, J=6.44 Hz, 1 H), 7.61 (d, J=9.49 Hz, 1 H), 9.76 (s, 1 H). MS (ESI) positive ion; m/z 468 (M+Na)⁺, 446 (M+H)⁺: negative ion; m/z 444 (M−H)⁻.

### EXAMPLE 14

### 3-(2,6-dichlorophenyl)-N-[4-(diethylamino)phenyl]-5-isopropylisoxazole-4-carboxamide

### EXAMPLE 14A

### 3-(2,6-Dichloro-phenyl)-5-isopropyl-isoxazole-4-carboxylic acid methyl ester

To a solution of methyl isobutyrylacetate (288 mg, 2.00 mmol), in THF (1.5 mL) was added NaOMe (4 mL, 2 mmol, 0.5 M in MeOH). The mixture was stirred for 30 min. 2,6-Dichloro-N-hydroxybenzenecarboximidoyl chloride (449 mg, 2.00 mmol) in THF (2 mL) was then added and the mixture stirred overnight. The titled compound (349 mg, 56%) was obtained following column chromatography (0-25% ethyl acetate in hexanes).

### EXAMPLE 14B

### 3-(2,6-Dichloro-phenyl)-5-isopropyl-isoxazole-4-carboxylic acid

Compound 14A was stirred in aqueous NaOH (4 mL, 2M) for 24 hours. HCl (20 mL, 1M) was added and the reaction mixture was extracted in ethyl acetate (20 mL×2) to provide the titled compound 14B (308 mg, 89%).

### EXAMPLE 14C

### 3-(2,6-dichlorophenyl)-N-[4-(diethylamino)phenyl]-5-propylisoxazole-4-carboxamide

Compound 14B (308 mg, 1.08 mmol), N,N-diethyl-1,4-phenylenediamine (249 mg, 1.52 mmol), TBTU (485 mg, 1.51 mmol), and i-Pr₂NEt (196 mg, 1.52 mmol) were dissolved in DMF (2 mL). The mixture was stirred for 14 hours and prurified by reverse phase HPLC (0-70% acetonitrile in 0.1% aqueous NH₄OAc) providing the titled compound 14C (220 mg, 45.7%). ¹H NMR (300 MHz, DMSO-d6) δ 1.04 (t, J=6.95 Hz, 6 H), 1.37 (d, J=7.12 Hz, 6 H), 3.28 (q, J=6.78 Hz, 4 H), 3.62 (m, J=6.78 Hz, 1 H), 6.59 (d, J=9.16 Hz, 2 H), 7.27 (d, J=8.82 Hz, 2 H), 7.53 (dd, J=9.49, 6.44 Hz, 1 H), 7.60 (d, J=6.78 Hz, 1 H), 7.61 (d, J=9.16 Hz, 1 H), 9.70 (s, 1 H). MS (ESI) positive ion; m/z 468 (M+Na)+, 446 (N+H)⁺, 417 (M-Et)⁺: negative ion; m/z 444 (M−H)⁻.

### EXAMPLE 15

### 3-(2,6-dichlorophenyl)-N-[4-(diethylamino)phenyl]-5-(2-furyl)isoxazole-4-carboxamide

The titled compound was prepared according to the procedure described in Example 14, substituting 3-furan-2-yl-3-oxo-propionic acid methyl ester for methyl isobutyrylacetate used in Example 14A. ¹H NMR (300 MHz, DMSO-d6) δ 1.05 (t, J=6.95 Hz, 6 H), 3.28 (q, J=7.12 Hz, 4 H), 6.59 (d, J=9.16 Hz, 2 H), 6.91 (dd, J=2.03, 0.68 Hz, 1 H), 7.30 (d, J=8.82 Hz, 2 H), 7.56 (dd, J=9.49, 6.10 Hz, 1 H), 7.631 (d, J=6.44, 1 H), 7.64 (d, J=8.81, 1 H), 7.94 (t, J=1.87 Hz, 1 H), 8.45 (dd, J=1.3, 0.68, 1 H), 10.09 (s, 1 H). MS (ESI) positive ion; m/z 492 (M+Na)⁺, 470 (M+H)⁺, 441 (M-Et)+: negative ion; m/z 468 (M−H)⁻.

### EXAMPLE 16

### 3-(2,6-dichlorophenyl)-N-[4-(diethylamino)-2-methoxyphenyl]-5-methylisoxazole-4-carboxamide

### EXAMPLE 16A

### 5-Diethylamino-2-nitro-phenol

A mixture of 5-chloro-2-nitroanisole (375 mg, 2.0 mmol), N,N-diethylamine (302 μL, 3.0 mmol), diisopropylethylamine (523 uL, 3.0 mmol) and DMF (2 mL) in a sealed tube was heated in a microwave oven at 200° C. for 15 minutes. It was partitioned between ethyl acetate and water (40 mL, 1:1). The organic phase was washed with brine (×3), dried (MgSO₄), filtered and concentrated under reduced pressure. The residue was purified on silica gel with hexane/ethyl acetate (4/1) to provide the titled compound (50 mg). ¹H NMR (300 MHz, DMSO-d₆) δ 11.10 (s, 1H), 7.86 (d, J=9.8 Hz, 1H), 6.45 (dd, J, =2.7 Hz, J₂=9.8 Hz, 1H), 6.17 (d, J=2.7 Hz, 1H), 3.46 (q, J=7.1 Hz, 4H), 1.13 (t, J=71 Hz, 6H); MS (ESI(+)) m/e 211 (M+H)⁺.

### EXAMPLE 16B

### Diethyl-(3-methoxy-4-nitro-phenyl)-amine

To the mixture of the phenol from Example 16A (21 mg, 0.1 mmol), methanol (6 μL, 0.15 mmol), and triphenylphosphine (34 mg, 0.13 mmol) in THF (0.5 ML) was added DEAD (20 μL, 0.13 mmol). The reaction mixture was stirred at ambient temperature for 30 minutes and then concentrated under reduced pressure. The concentrate was purified by flash chromatography on silica gel with hexane/ethyl acetate (2:1) to provide 19 mg titled compound as yellow oil. ¹H NMR (300 MHz, DMSO-d₆) δ 7.90 (d, J=9.5 Hz, 1H), 6.35 (dd, J₁=2.7 Hz, J₂=9.5 Hz, 1H), 6.20 (d, J=2.7 Hz, 1H), 3.90 (s, 3H), 3.47 (q, J=7.1 Hz, 4H), 1.14 (t, J=7.1 Hz, 6H); MS (ESI(+)) m/e 225 (M+H)⁺.

### EXAMPLE 16C

### N,N-diethyl-2-methoxy-1,4-phenylenediamine

The material from Example 16B (14 mg) and 10% Pd—C (5 mg) in methanol (2 mL) plus methanol with saturated HCl (0.5 mL) was stirred under an atmosphere of hydrogen at ambient temperature for 1 hour to provide the tilted compound 15 mg. MS (ESI(+)) m/e 195 (M+H)⁺

### EXAMPLE 16D

### 3-(2,6-dichlorophenyl)-N-[4-(diethylamino)-2-methoxyphenyl]-5-methylisoxazole-4-carboxamide

The titled compound was prepared according to the procedure described in Example 1, substituting N,N-diethyl-2-methoxy-1,4-phenylenediamine for N,N-diethyl-1,4-phenylenediamine. ¹H NMR (300 MHz, DMSO-d₆) δ 8.06 (s, 1H), 7.76-7.63 (m, 4H), 6.21-6.14 (m, 2H), 3.62 (s, 3H), 3.29 (q, J=7.1 Hz, 4H), 2.79 (s, 3H), 1.05 (t, J=7.1 Hz, 6H); MS (ESI(+)) m/e 448 (M+H)⁺.

### EXAMPLE 17

### N-{4-[tert-butyl(ethyl)amino]phenyl}-3-(2,6-dichlorophenyl)-5-methylisoxazole-4-carboxamide

### EXAMPLE 17A

### N-tert-butyl-N-ethyl-benzene-1,4-diamine

To a DMF (5 mL) solution of tert-butyl-(4-nitro-phenyl)-amine (510 mg, 2.6 mmol) was added NaH (50%, 150 mg, 3.1 mmol). Ethyl iodide (470 mg, 3.0 mmol) was added after 5 minutes and the mixture was allowed to stir at room temperature for 16 hours. The reaction mixture was quenched with aqueous NH₄Cl solution (10 mL) and extracted with ethyl acetate (3×20 mL). The combined organic extracts were dried over MgSO₄, filtered and concentrated under reduced pressure. The residue was purified using column chromatography (hexanes:ethyl acetate=10:1) to provide t-butyl-ethyl-(4-nitro-phenyl)-amine (230 mg, 40%). This material was dissolved in MeOH (5 mL) followed by addition of Pd/C (5%, 10 mg). The mixture was stirred under an atmosphere of H₂ for 16 hours. Pd/C was filtered off and the filtrate was concentrated under reduced pressure to provide N-t-butyl-N-ethyl-benzene-1,4-diamine (200 mg, 100%).

### EXAMPLE 17B

### N-{4-[tert-butyl(ethyl)amino]phenyl}-3-(2,6-dichlorophenyl)-5-methylisoxazole-4-carboxamide

This title compound was prepared according to the procedure described in Example 1, substituting the N,N-diethylphenylene diamine from Example 1 for N-tert-butyl-N-ethyl-benzene-1,4-diamine from Example 17A. ¹H NMR (300 MHz, DMSO-d₆) δ 10.10 (s, 1H), 7.60 (d, J=9.0 Hz, 1H), 7.60 (d, J=6.9 Hz, 1H), 7.54 (dd, J=6.6, 9.9 Hz, 1H), 7.44 (d, J=9.0 Hz, 2H), 7.06 (d, J=8.7 Hz, 2H), 3.02 (q, J=6.9 Hz, 2H), 2.73 (s, 3H), 1.02 (s, 9H), and 0.72 (t, J=6.9 Hz, 3H). MS (ESI) positive ion: 446 (M+H)+, 448 (M+H)⁺; negative ion: 444 (M−H)⁻, 446 (M−H)⁻.

### EXAMPLE 18

### 3-(2,6-dichlorophenyl)-N-[4-(diethylamino)-2-hydroxyphenyl]-5-methylisoxazole-4-carboxamide

To the solution of methyl ether from Example 16D (8.5 mg, 0.019 mmol) in 0.5 mL dichloromethane was added 1M BBr₃ in dichloromethane dropwise at room temperature. The reaction mixture was stirred for 30 minutes, diluted with saturated NaHCO₃ (2 mL) and extracted with ethyl acetate (2 mL). The organic phase was purified on silica gel with hexane/ethyl acetate (2/1) to provide the titled compound (5 mg). ¹H NMR (300 MHz, DMSO-d₆) δ 9.28 (s, 1H), 8.28 (s, 1H), 7.67-7.46 (m, 4H), 6.12-6.03 (m, 2H), 3.22 (q, J=7.1 Hz, 4H), 2.79 (s, 3H), 1.04 (t, J=7.1 Hz, 6H); MS (ESI(+)) m/e 434 (M+H)⁺.

### EXAMPLE 19

### N-{4-[(2-chloroethyl)(ethyl)amino]phenyl}-3-(2,6-dichlorophenyl)-5-methylisoxazole-4-carboxamide

### EXAMPLE 19A

### Ethyl-(4-nitro-phenyl)-carbamic acid tert-butyl ester

To a solution of N-ethyl-4-nitroaniline (5.03 g, 30 mmol) in DMF (380 mL) was added sodium hydride (1.81 g, 45 mmol, 60% dispersion in mineral oil). The mixture was stirred until hydrogen evolution ceased. Di-tert-butyl-dicarbonate (9.85 g, 45 mmol) was added and the reaction was stirred for 2 hours. The mixture was extracted in ethyl acetate (250 mL) over 225 mL H₂O. The organic layer was washed with water (200 mL, 3×). The titled compound 19A (7.30 g, 91.5%) was obtained as a yellow solid.

### EXAMPLE 19B

### (4-Amino-phenyl)-ethyl-carbamic acid tert-butyl ester

To compound 19A (7.30 g, 27.4 mmol) and Pd/C (1.02 g) was added ethyl acetate (80 mL) under a nitrogen atmosphere. The flask was charged with hydrogen and stirred for 4 hours. The mixture was filtered and the solvent removed under reduced pressure to provide the titled compound (6.42 g, 99.1%).

### EXAMPLE 19C

### (4-{[3-(2,6-Dichloro-phenyl)-5-methyl-isoxazole-4-carbonyl]-amino}-phenyl)-ethyl-carbamic acid tert-butyl ester

To a solution of 3-(2,6-dichlorophenyl)-5-methylisoxazole-4-carboxylic acid (1.00 g, 3.68 mmol) and N butoxycarbonyl-N-ethyl-1,4-phenylenediamine compound 19B (0.868 g, 3.68 mmol) in DMF (6 mL) were added Et₃N (0.66 g, 5.15 mmol) and TBTU (1.65 g, 5.15 mmol). After stirring for 16 hours. The reaction was washed with water (50 mL) and extracted with ethyl acetate (75 mL). Compound 19C (1.17 g, 64.9%) was obtained after separation by column chromatography (5-25% ethyl acetate in hexanes).

### EXAMPLE 19D

### 3-(2,6-Dichloro-phenyl)-5-methyl-isoxazole-4-carboxylic acid (4-ethylamino-phenyl)-amide

To compound 19C (1.14 g, 2.33 mmol) was added HCl in dioxane (20 mL, 4M) at 0° C. The reaction mixture was warmed to room temperature and stirred for 2 hours. The solvent was removed under reduced pressure providing the titled compound 19D (0.895 g, 98.5%).

### EXAMPLE 19E

### N-{4-[(2-chloroethyl)(ethyl)amino]phenyl}-3-(2,6-dichlorophenyl)-5-methylisoxazole-4-carboxamide

Compound 19D (80 mg, 0.21 mmol) and 3-chloroacetaldehyde (5.3 mg, 0.41 mmol) were added to a buffered solution of NaOAc (88 mg) and acetic acid (88 uL) in MeOH (1 mL). After stirring for 15 minutes, NaBH₃CN (32 mg, 0.52 mmol) was added and the mixture stirred for 14 hours. Compound 19E (36 mg, 38%) was obtained after reverse phase HPLC (0-70% acetonitrile in 0.1% aqueous NH₄Cl). ¹H NMR (300 MHz, DMSO-d6) δ 1.05 (t, J=6.95 Hz, 3 H), 2.71 (s, 3 H), 3.37 (q, J=7.12 Hz, 2 H), 3.57 (t, J=6.27 Hz, 2 H), 3.68 (t, J=6.27 Hz, 2 H), 6.64 (d, J=8.82 Hz, 2 H), 7.34 (d, J=8.82 Hz, 2 H), 7.53 (dd, J=9.55, 6.27 Hz, 1 H), 7.60 (d, J=6.44, 1 H), 7.61 (d, J=9.16, 1 H), 9.82 (s, 1 H). MS (ESI) positive ion; m/z 452 (M+H)⁺: negative ion; m/z 450 (M−H)⁻.

### EXAMPLE 20

### 3-(2,6-dichlorophenyl)-N-{4-[ethyl(propyl)amino]phenyl}-5-methylisoxazole-4-carboxamide

The titled compound was prepared according to the procedure described in Example 19, substituting n-propionaldehyde for 3-chloroacetaldehyde. ¹H NMR (300 MHz, DMSO-d6) δ 0.86 (t, J=7.29 Hz, 3 H), 1.03 (t, J=6.95 Hz, 3 H), 1.50 (sextet, J=7.5 Hz, 2 H), 2.71 (s, 3 H), 3.16 (t, J=7.5 Hz, 2 H), 3.30 (q, J=7.8, 2 H), 6.57 (d, J=9.16 Hz, 2 H), 7.30 (d, J=8.82 Hz, 2 H), 7.53 (dd, J=9.49, 6.10 Hz, 1 H), 7.60 (d, J=6.9 Hz, 1 H), 7.61 (d, J=9.3, 1H), 9.76 (s, 1 H). MS (ESI) positive ion; m/z 432 (M+H)⁺: negative ion; m/z 430 (M−H)⁻.

### EXAMPLE 21

### N-{4-[butyl(ethyl)amino]phenyl}-3-(2,6-dichlorophenyl)-5-methylisoxazole-4-carboxamide

The titled compound was prepared according to the procedure described in Example 19, substituting n-butaldehyde for 3-chloroacetaldehyde. ¹H NMR (300 MHz, DMSO-d6) δ 0.90 (t, J=7.29 Hz, 3 H), 1.03 (t, J=6.95 Hz, 3 H), 1.29 (sextet, J=7.5 Hz, 2 H), 1.46 (sextet, J=7.5 Hz, 2 H), 2.71 (s, 3 H), 3.20 (t, J=8.1 Hz, 2 H), 3.299 (q, J=7.2 Hz, 2 H), 6.57 (d, J=9.16 Hz, 2 H), 7.29 (d, J=9.16 Hz, 2 H), 7.53 (dd, J=9.66, 6.27 Hz, 1 H), 7.60 (d, J=6.9 Hz, 1 H), 7.61 (d, J=9.6 Hz, 1 H), 9.76 (s, 1 H). MS (ESI) positive ion; m/z 446 (M+H)⁺: negative ion; m/z 444 (M−H)⁻.

### EXAMPLE 22

### 3-(2,6-dichlorophenyl)-N-[4-(diethylamino)-2-methylphenyl]-5-propylisoxazole-4-carboxamide

The titled compound was prepared according to the procedure described in Example 14, substituting the 3-(2,6-Dichloro-phenyl)-5-propyl-isoxazole-4-carboxylic acid from Example 13 for the acid from Example 14B, and N,N-diethyl-2-methyl-1,4-phenylenediamine for the N,N-diethylphenylene diamine. ¹H NMR (300 MHz, DMSO-d6) δ 0.96 (t, J=7.29 Hz, 3 H), 1.05 (t, J=7.2 Hz, 6 H), 1.80 (sextet, J=7.5 Hz, 2 H), 2.04 (s, 3 H), 3.15 (t, J=7.29 Hz, 2 H), 3.28 (q, J=7.24 Hz, 4 H), 6.45 (d, J=8.48 Hz, 1 H), 6.47 (s, 1 H), 6.93 (d, J=8.48 Hz, 1 H), 7.53 (dd, J=9.32, 6.61 Hz, 1 H), 7.61 (d, J=7.24 Hz, 1 H), 7.62 (d, J=9.30 Hz, 1 H), 9.17 (s, 1 H). MS (ESI) positive ion; m/z 461 (M+H)⁺: negative ion; m/z 459 (M−H)⁻, 424 (M-Cl)⁻.

### EXAMPLE 23

### 3-(2,6-dichlorophenyl)-N-[4-(diethylamino)-2-(piperidin-1-ylcarbonyl)phenyl]-5-methylisoxazole-4-carboxamide

### EXAMPLE 23A

### (5-Diethylamino-2-nitro-phenyl)-piperidin-1-yl-methanone

The titled compound was prepared according to the procedure described in Example 1, substituting 5-Diethylamino-2-nitro-benzoic acid for 3-(2,6-Dichloro-phenyl)-5-methyl-isoxazole-4-carboxylic acid and piperidine for N,N-diethyl-1,4-phenylenediamine. MS (APCI(+)) m/e 305 (M+H)⁺.

### EXAMPLE 23B

### (2-Amino-5-diethylamino-phenyl)-piperidin-1-yl-methanone

The titled compound was prepared according to the procedure described in Example 16C, substituting (5-Diethylamino-2-nitro-phenyl)-piperidin-1-yl-methanone for Diethyl-(3-methoxy-4-nitro-phenyl)-amine.

### EXAMPLE 23C

### 3-(2,6-dichlorophenyl)-N-[4-(diethylamino)-2-(piperidin-1-ylcarbonyl)phenyl]-5-methylisoxazole-4-carboxamide

The titled compound was prepared according to the procedure described in Example 1, substituting (2-amino-5-diethylamino-phenyl)-piperidin-1-yl-methanone for N,N-diethyl-1,4-phenylenediamine. ¹H NMR (300 MHz, DMSO-d₆) δ 9.18 (s, 1H), 7.63-7.52 (m, 3H), 7.21 (d, J=8.2 Hz, 1H), 6.66 (dd, J=8.2 Hz, J=3.1 Hz, 1H), 6.42 (d, J=3.1 Hz, 1H), 3.57-3.10 (m, 8H), 2.73 (s, 3H), 1.59-1.28 (m, 6H), 1.06 (t, J=6.8 Hz, 6H). MS (ESI(+)) m/e 529 (M+H)⁺.

### EXAMPLE 24

### 3-(2,6-dichlorophenyl)-N-[4-(diethylamino)phenyl]-5-(2-phenylethyl)isoxazole-4-carboxamide

The titled compound was prepared according to the procedure described in Example 3, substituting benzyl bromide for the allyl iodide used in Example 3A. ¹H NMR (300 MHz, CDCl₃) δ 7.48 (dd, J=6.8, 2.4 Hz, 1H), 7.47 (br s, 1H), 7.41 (dd, J=9.5, 6.4 Hz, 1H), 7.31-7.17 (m, 5H), 6.94 (d, J=9.2 Hz, 2H), 6.54 (d, J=9.2 Hz, 2H), 6.40 (s, 1H), 3.51 (t, J=7.6 Hz, 2H), 3.29 (q, J=7.1 Hz, 4H), 3.18 (t, J=7.6 Hz, 2H), 1.10 (t, J=7.1 Hz, 6H); MS )ESI) m/e 508, 510, 512 (M+H)⁺.

### EXAMPLE 25

### 3-(2,6-dichlorophenyl)-N-[4-(diethylamino)-2-ethylphenyl]-5-methylisoxazole-4-carboxamide

The titled compound was prepared according to the procedure described in Example 1, substituting N,N-diethyl-2-ethyl-1,4-phenylenediamine for N,N-diethyl-1,4-phenylenediamine. ¹H NMR (300 MHz, DMSO-d₆) δ 9.17 (s, 1H), 7.63-7.50 (m, 3H), 6.93 (d, J=9.5 Hz, 1H), 6.49-6.42 (m, 2H), 3.29 (q, J=7.1 Hz, 4H), 2.76 (s, 3H), 2.44 (q, J=7.8 Hz, 2H), 1.05-1.01(m, 9H); MS (ESI(+)) m/e 446 (M+H)⁺.

### EXAMPLE 26

### 3-(2,6-dichlorophenyl)-N-[4-(diethylamino)phenyl]-5-[3-(dimethylamino)-3-oxopropyl]isoxazole-4-carboxamide

### EXAMPLE 26A

### 3-(2,6-dichlorophenyl)-5-[3-(dimethylamino)-3-(oxopropyl]-4-ethyl carboxyisoxazole

A solution of sodium ethoxide (75 mg, 1.1 mmol) in ethanol (1 ml) was added to a solution of 5-dimethylcarbamoyl-3-oxo-pentanoic acid ethyl ester (215 mg, 1.0 mmol) in ethanol (6 ml). The mixture was stirred at room temperature for 15 minutes, dichloro-N-hydroxybenzenecarboximidoyl chlorode (247 mg, 1.1 mmol) was added. The reaction mixture was stirred for another 1 hour and the solvent removed under reduced pressure. To the residue were added methylene chloride and water (15 ml, 1:1). The organic layer was washed with brine, dried over magnesium sulfate, filtered and concentrated under reduced pressure and purified by column chromatography using hexane and ethyl acetate (1:1) as eluent to provide the titled compound (380 mg, 98%) as colorless oil. ¹HNMR (CDCl₃, 300 MHz), δ 7.43-7.31 (m, 3H, Ar—H), 4.12(q, J=7.5 Hz, 2H, OCH₂CH₃), 3.55 (t, J=9.0 Hz, 2H, CH₂CH₂), 2.88 (t, J=9.0 Hz, 2H, CH₂CH₂), 3.05 (s, 2H, NCH₃), 2.99 (s, 3H, NCH₃), 1.04 (t, J=7.5 Hz, 2H, CH₂CH₃). MS (ESI) m/e 385.0 (M)⁺.

### EXAMPLE 26B

### 3-(2,6-dichlorophenyl)-N-[4-(diethylamino)phenyl]-5-r3-(dimethylamino)-3-oxopropyl]-isoxazole-4-carboxamide

Ethylmagnesium bromide (0.17 ml, 0.52 mmol, 3M in ethyl ether) was added to a solution of N,N-diethyl-1,4-phenylene diamine (85 mg, 0.52 mmol) in THF (5 ml) at 0° C. The mixture was stirred at room temperature for 15 minutes, then 3-(2,6-dichlorophenyl)-5-[3-(dimethylamino)-3-(oxopropyl]-4-ethyl carboxyisoxazole from Example 26A (100 mg, 0.26 mmol) in THF (2 ml) was added. The reaction mixture was stirred for overnight at room temperature and the solvent removed under reduced pressure. To the residue was added methylene chloride and aqueous of NH₄Cl (10 ml, 1:1). The organic layer was washed with brine, dried over magnesium sulfate, filtered and concentrated under reduced pressure and purified by prep-HPLC to provide 81 mg (62%) of the titled compound. ¹HNMR (CDCl₃, 300 MHz), δ 10.82 (br s, 1H,CONH), 7.55 (d, J=9.0 Hz, 2H, Ar′—H), 6.63 (d, J=9.0 Hz, 2H, Ar′—H), 7.39-7.28 (m, 3H, Ar—H), 3.34 (t, J=6.0 Hz, 2H, CH₂CH₂), 3.01 (t, J=6.0 Hz, 2H, CH₂CH₂), 3.30 (q, J=6.0 Hz, 4H, NCH₂CH₃), 3.07 (s, 3H, 2NCH₃), 3.00 (s, 3H, 2NCH₃), 1.11 (t, J=6.0 Hz, 6H, 2 NCH₂CH₃). MS (ESI) m/e 503.0, 501.1 (M)⁺.

### EXAMPLE 27

### 3-(2,6-dichlorophenyl)-N-[4-(diethylamino)phenyl]-5-[2-(1,3-dioxan-2-yl)ethyl]isoxazole-4-carboxamide

The titled compound was prepared according to the procedure described in Example 26, substituting 5-[1,3]dioxan-2-yl-3-oxo-pentanoic acid ethyl ester for the 5-dimethylcarbamoyl-3-oxo-pentanoic acid ethyl ester used in Example 26A. ¹HNMR (CDCl₃, 300 MHz), δ 7.79 (br s, 1H,CONH), 7.48-7.35 (m, 3H, Ar—H), 7.20 (d, J=9.0 Hz, 2H, Ar′—H), H), 6.61 (d, J=9.0 Hz, 2H, Ar′—H), 4.62 (t, J=6.0 Hz, 1H, O—CH—O), 4.10 (q, J=6.0 Hz, 2H, OCH₂CH₃), 3.74 (dt, J₁=12 Hz, J₂=3.0 Hz, 2H, OCH₂CH₂), 3.30 (q, J=6.0 Hz, 4H, 2NCH₂CH₃ and 2H for CH₂CH₂), 2.24 (dq, J₁=6.0 Hz, J₂=3.0 Hz, 2H, CHCH₂CH₂), 2.07 (m, 2H, CH₂CH₂CH₂), 1.11 (t, J=6.0 Hz, 6H, 2 NCH₂CH₃). MS (ESI) m/e 518.1, 516.1 (M)⁺.

### EXAMPLE 28

### 5-[4-(acetylamino)butyl]-3-(2,6-dichlorophenyl)-N-[4-(diethylamino)phenyl]isoxazole-4-carboxamide

To a stirred solution of amine from Example 11 (25 mg, 0.05 mmol) in dichloromethane was added Et₃N (11 μL, 0.075 mmol) and acetyl chloride (4 μL, 0.08 mmol). After 30 minutes at ambient temperature, the volatile solvent was removed under reduced pressure and the residue was purified on a Gilson preparative HPLC to provide the titled compound (15 mg, 58%). ¹H NMR (300 MHz, CDCl₃) δ 7.54 (d, J=9.5 Hz, 1H), 7.54 (d, J=6.8 Hz, 1H), 7.47 (dd, J=9.5, 6.1 Hz, 1H), 7.04 (d, J=8.8 Hz, 2H), 6.85 (br s, 1H), 6.61 (br d, 2H), 5.98 (br s, 1H), 3.40-3.20 (m, 6H), 2.10-2.02 (m, 2H), 1.97 (s, 3H), 1.96-1.86 (m, 2H), 1.72-1.58 (m, 2H), 1.11 (t, J=7.12 Hz, 6H). MS (ESI) 517, 519 (M+H)⁺.

### EXAMPLE 29

### 3-(2,6-dichlorophenyl)-N-[4-(diethylamino)phenyl]-5-{4-[(methylsulfonyl)amino]butyl}isoxazole-4-carboxamide

The titled compound was prepared according to the procedure described in Example 28, substituting methane sulfonyl chloride for acetyl chloride used in Example 28. ¹H NMR (300 MHz, CDCl₃) δ 7.54 (d, J=9.5 Hz, 1H), 7.54 (d, J=6.8 Hz, 1H), 7.47 (dd, J=9.5, 6.1 Hz, 1H), 7.04 (d, J=8.8 Hz, 2H), 6.85 (br s, 1H), 6.61 (br d, 2H), 5.98 (br s, 1H), 3.40-3.20 (m, 6H), 2.85 (s, 3H), 2.56-2.44 (m, 2H), 1.97 (s, 3H), 1.96-1.86 (m, 2H), 1.72-1.58 (m, 2H), 1.11 (t, J=7.12 Hz, 6H). MS (ESI) 553, 555 (M+H)⁺.

### EXAMPLE 30

### 3-(2,6-dichlorophenyl)-N-[4-(diethylamino)phenyl]-5-[3-(1,3-dioxan-2-yl)propyl]isoxazole-4-carboxamide

The titled compound was prepared according to the procedure described in Example 26, substituting 6-[1,3]dioxan-2-yl-3-oxo-hexanoic acid ethyl ester for the 5-dimethylcarbamoyl-3-oxo-pentanoic acid ethyl ester used in Example 26A. Light brown oil;

¹H NMR (300 MHz, CDCl₃) δ 7.53 (d, J=2.4 Hz, 1H), 7.50 (br s, 1H), 7.44 (dd, J=9.5, 6.1 Hz, 1H), 7.02 (d, J=9.2 Hz, 2H), 6.78 (s, 1H), 6.56 (d, J=9.2 Hz, 2H), 4.60 (t, J=5.1 Hz, 1H), 4.14-4.05 (m, 2H), 3.81-3.67 (m, 2H), 3.29 (q, J=7.1 Hz, 4H), 1.94-2.08 (m, 2H), 1.80-1.69 (m, 2H), 1.36-1.28 (m, 2H), 1.10 (t, J=7.1 Hz, 6H); MS (ESI) m/e 532, 534, 536 (M+H)⁺.

### EXAMPLE 31

### 3-(2,6-dichlorophenyl)-N-[4-(diethylamino)phenyl]-5-(3-hydroxypropyl)isoxazole-4-carboxamide

### EXAMPLE 31A

### 3-(2,6-Dichloro-phenyl)-5-(3-oxo-propyl)-isoxazole-4-carboxylic acid (4-diethylamino-phenyl)-amide

The solution of 3-(2,6-dichlorophenyl)-N-[4-(diethylamino)phenyl]-5-[2-(1,3-dioxan-2-yl)ethyl]isoxazole-4-carboxamide from Example 27 in 30% acetic acid aqueous (10 ml) was heated to reflux for 6 hours, extracted with methylene chloride two times. The solvent was removed under reduced pressure to provide 44 mg (95%) of the titled compound 31A was obtained which used in next step without purification. ¹HNMR (CDCl₃, 300 MHz), δppm, 9.88 (s, 1H, —CHO), 8.05 (br s, 1H,CONH), 7.48-7.37 (m, 3H, Ar—H), 7.20 (d, J=9.0 Hz, 2H, Ar′—H), 6.61 (d, J=9.0 Hz, 2H, Ar′—H), 3.46 (t, J=6.0 Hz, 2H, CH₂CH₂), 3.19 (t, J=6.0 Hz, 2H, CH₂CH₂), 3.30 (q, J=6.0 Hz, 4H, 2NCH₂CH₃), 1.11 (t, J=6.0 Hz, 2 NCH₂CH₃).

MS, M⁺460.0, 458.0.

### EXAMPLE 31B

### 3-(2,6-dichlorophenyl)-N-[4-(diethylamino)phenyl]-5-(3-hydroxypropyl)isoxazole-4-carboxamide

To a solution of 3-(2,6-dichloro-phenyl)-5-(3-oxo-propyl)-isoxazole-4-carboxylic acid (4-diethylamino-phenyl)-amide from Example 31 A in 2 ml methanol, sodium borohydride (1.6 mg, 0.043 mmol) was added at 0° C. The mixture was then stirred at room temperature for 0.5 hour, 2N HCl (1 ml) was added to the mixture, then water and methylene chloride (20 ml, 1:1) was added. The organic layer was washed with aqueous sodium bicarbonate and brine (2×), dried over magnesium sulfate, filtered, concentrated under reduced pressure and the residue purified on column of silica gel using hexane and ethyl acetate as eluent to provide the titled compound 36 mg (90%). ¹HNMR (CDCl₃, 300 MHz), δppm, 7.53-7.42 (m, 3H, Ar—H), 7.21 (br s, 1H,CONH), 7.06 (d, J=9.0 Hz, 2H, Ar′—H), 6.56 (d, J=9.0 Hz, 2H, Ar′—H), 3.63 (q, J=6.0 Hz, 2H, CH₂CH₂OH), 3.48 (t, J=6.0 Hz, 1H, CH₂OH), 3.36 (t, J=6.0 Hz, 2H, —CH₂CH₂), 3.30 (q, J=6.0 Hz, 4H, 2NCH₂CH₃), 2.10 (m, 2H, CH2CH2CH₂), 1.11 (t, J=6.0 Hz, 6H, 2 NCH₂CH₃); MS (ESI) m/e (M)⁺462.0, 460.0.

### EXAMPLE 32

### 3-(2,6-dichlorophenyl)-N-[4-(diethylamino)phenyl]-5-(2-hydroxy-2-phenylethyl)isoxazole-4-carboxamide

The titled compound was prepared according to the procedure described in Example 3, substituting benzaldehyde for the allyl iodide used in Example 3A. ¹H NMR (300 MHz, CDCl₃) δ 8.11 (d, J=7.1 Hz, 1H), 7.70-7.32 (m, 8H), 7.16 (d, J=8.8 Hz, 1H), 6.65 (d, J=8.8 Hz, 2H), 4.93 (br m, 1H), 3.65-3.44 (m, 2H), 3.29 (q, J=7.1 Hz, 4H), 1.10 (t, j=7.1 Hz, 6H); MS (ESI) m/e 524, 526, 528 (M+H)⁺.

### EXAMPLE 33

### 3-(2,6-dichlorophenyl)-N-[4-(diethylamino)phenyl]-5-(2-tetrahydro-2H-pyran-2-ylethyl)isoxazole-4-carboxamide

The titled compound was prepared according to the procedure described in Example 26, substituting 3-(2-tetrahydro-2H-pyranyl)-propionyl ethyl acetate for the 5-dimethylcarbamoyl-3-oxo-pentanoic acid ethyl ester used in Example 26A. ¹HNMR (CDCl₃, 300 MHz), δ 9.52 (br s, 1H,CONH), 7.72-7.39 (m, 7H, Ar—H and Ar′—H), 3.97-3.72 (m, 3H, CHOCH₂), 3.36-3.10 (m, 8H, NCH₂CH₂ and 2 CH₂CH₃), 1.84-1.58 (m, 6H, CH₂CH₂CH₂),1.11 (t, J=7.12 Hz, 6H, 2CH₂CH₃); MS (ESI) m/e 516.0, 514.0 (M)⁺.

### EXAMPLE 34

### 3-(2,6-dichlorophenyl)-N-[4-(diethylamino)phenyl]-5-(tetrahydro-2H-pyran-4-ylmethyl)isoxazole-4-carboxamide

The titled compound was prepared according to the procedure described in Example 26, substituting ethyl 2-(4-tetrahydro-2H-pyranyl)-aceto acetate for the 5-dimethylcarbamoyl-3-oxo-pentanoic acid ethyl ester used in Example 26A. ¹HNMR (CDCl₃, 300 MHz), δ 7.55-7.45 (m, 3H, Ar—H), 7.00 (d, J=9.0 Hz, 2H, Ar—H), 6.56 (d, J=9.0 Hz, 2H, Ar′—H), 6.70 (br s, 1H,CONH), 3.98 (d, J=9.0 Hz, 2H, CH₂O), 3.41 (t, J=9.0 Hz, 2H, CH₂O), 3.29 (q, J=7.0 Hz, 2H, —CH₂CH₃), 3.23 (d, J=7.0 Hz, 2H, CH₂), 2.2 (m, 1H, CH), 1.67-1.48 (m, 4H, 2CH₂), 1.10 (t, J=7.0 Hz, 6H, 2CH₃); MS (ESI) m/e 502.1, 504.0 (M)⁺.

### EXAMPLE 35

### 5-butyl-3-(2,6-dichlorophenyl)-N-[4-(diethylamino)phenyl]isoxazole-4-carboxamide

The titled compound was prepared according to the procedure described in Example 14, substituting 3-oxo-heptanoic acid methyl ester for the methyl isobutyrylacetate used in Example 14A. ¹H NMR (300 MHz, DMSO-d6) δ 0.88 (t, J=7.29 Hz, 3 H), 1.04 (t, J=7.12 Hz, 6 H), 1.33 (sextet, J=7.46 Hz, 2 H), 1.71 (pentet, J=7.46 Hz, 2 H), 3.12 (t, J=7.29 Hz, 2 H), 3.28 (q, J=7.12 Hz, 4 H), 6.59 (d, J=9.16 Hz, 2 H), 7.28 (d, J=8.82 Hz, 2 H), 7.53 (dd, J=9.49, 6.10 Hz, 1 H), 7.60 (d, J=6.10 Hz, 1 H), 7.61 (d, J=9.49 Hz, 1 H), 9.76 (s, 1 H).

### EXAMPLE 36

### 3-(2,6-dichlorophenyl)-5-[2-(1,3-dioxan-2-yl)ethyl]-N-{2-[(2-methyl-1,2,3,4-tetrahydroisoquinolin-1-yl)methyl]phenyl}isoxazole-4-carboxamide

The titled compound was prepared according to the procedure described in Example 27, substituting 2-(2-methyl-1,2,3,4-tetrahydro-isoquinolin-1-ylmethyl)-phenylamine for N,N-diethyl-1,4-phenylenediamine. ¹H NMR (300 MHz, DMSO-d₆) δ 10.67 (s, 1H), 7.59-6.96 (m, 11H), 4.54 (t, 1H), 4.00-3.92 (m, 2H), 3.88-3.82 (m, 1H), 3.74-3.61 (m, 2H), 3.29-2.68 (m, 8H), 2.30 (s, 3H), 2.04-1.25 (m, 4H); MS (ESI(+)) m/e 606 (M+H)⁺;

### EXAMPLE 37

### 3-(2,6-dichlorophenyl)-N-[4-(diethylamino)phenyl]-5-[(2-oxopyrrolidin-1-yl)methyl]isoxazole-4-carboxamide

The titled compound was prepared according to the procedure described in Example 26, substituting ethyl 3-(2-oxopyrrolidin-1-yl)-aceto acetate for the 5-dimethylcarbamoyl-3-oxo-pentanoic acid ethyl ester used in Example 26A. ¹HNMR (CDCl₃, 300 MHz), δ 10.17 (br s, 1H,CONH), 7.47-7.29 (m, 3H, Ar—H), 7.52 (d, J=9.15 Hz, 2H, Ar′—H), 6.61 (d, J=9.15 Hz, 2H, Ar′—H), 4.75 (s, 2H, CH₂), 3.83 (t, J=7.12 Hz, 2H, CH₂N), 3.31 (q, J=7.12 Hz, 2H, —CH₂CH₃), 2.51 (t, J=7.8 Hz, 2H, CH₂CO), 2.21(pentet, J=7.12 Hz, 2H, CH₂), 1.10 (t, J=7.12 Hz, 6H, 2CH₃); MS (ESI) m/e 501.1, 503.5 (M)⁺.

### EXAMPLE 38

### N-(2-{[(1,3-benzodioxol-5-ylmethyl)amino]carbonyl}phenyl)-3-(2,6-dichlorophenyl)-5-methylisoxazole-4-carboxamide

### EXAMPLE 38A

### 3-(2,6-Dichloro-phenyl)-5-methyl-isoxazole-4-carbonyl chloride

The suspension of 3-(2,6-Dichloro-phenyl)-5-methyl-isoxazole-4-carboxylic acid (8.17 g, 30.0 mmol) in thionyl chloride (40 mL) was heated to reflux for 1 hour. The mixture was concentrated under reduced pressure to provide the tilted compound 8.7 g. ¹H NMR (300 MHz, DMSO-d₆) δ 7.68-7.52 (m, 3H), 3.78 (s, 3H).

### EXAMPLE 38B

### 2-{[3-(2,6-Dichloro-phenyl)-5-methyl-isoxazole-4-carbonyl]-amino}-benzoic acid allyl ester

Saturated NaHCO₃ (12 mL) was added to a solution of 3-(2,6-dichloro-phenyl)-5-methyl-isoxazole-4-carbonyl chloride from Example 38A (1.3 g, 4.48 mmol) and 2-amino-benzoic acid allyl ester (721 mg, 4.07 mmol) in THF (12 mL). The reaction mixture was stirred at room temperature overnight. The mixture was partitioned between ethyl acetate and saturated NaHCO₃ (50 mL, 1:1), the separated organic phase was washed with brine (×3), dried (MgSO₄), filtered and concentrated under reduced pressure. The residue was purified on silica gel with hexane/ethyl acetate (4/1) to provide the titled compound (852 mg). ¹H NMR (300 MHz, DMSO-d₆) δ 10.78 (s, 1H), 8.29-8.26 (m, 1H), 7.96-7.92 (m, 1H), 7.65-7.53 (m, 4H), 7.25-6.98 (m, 1H), 6.07-5.93 (m, 1H), 5.43-5.25 (m, 2H), 4.75-4.71 (m, 2H), 2.85 (s, 3H); MS (ESI(+)) m/e 431 (M+H)⁺.

### EXAMPLE 38C

### 2-{[3-(2,6-Dichloro-phenyl)-5-methyl-isoxazole-4-carbonyl]-amino }-benzoic acid

To a solution of allyl ester from Example 38B (850 mg, 1.97 mmol) and Pd(PPh₃)₄ (68 mg, 0.059 mmol) in dichloromethane (10 mL) was added morpholine (344 μL, 3.94 mmol). The reaction mixture was stirred at room temperature under nitrogen atmosphere overnight, partitioned between ethyl acetate and 1N HCl (50 mL, 1:1). The separated organic phase was washed with brine, dried (MgSO₄), filtered and concentrated under reduced pressure to provide the titled compound (812 mg) as a pale yellow solid.

### EXAMPLE 38D

### N-(2-{[(1,3-benzodioxol-5-ylmethyl)amino]carbonyl}phenyl)-3-(2,6-dichlorophenyl)-5-methylisoxazole-4-carboxamide

A mixture of Example 38C (20 mg, 0.05 mmol), C-benzo[1,3]dioxol-5-yl-methylamine (15 mg, 0.10 mmol), HATU (38 mg, 0.10 mmol) and diisopropylethylamine (26 uL, 0.15 mmol) in DMF (500 uL) was stirred at room temperature overnight and then purified by reverse-phase HPLC with 5-100% CH₃CN/H₂O with 0.1% TFA to provide 11 mg of the titled compound. ¹H NMR (300 MHz, DMSO-d₆) δ 11.55 (s, 1H), 9.24 (t, J=5.8 Hz, 1H), 8.32 (d, J=8.4 Hz, 1H), 7.75 (d, J=6.9 Hz, 1H), 7.62-7.45 (m, 4H), 7.17 (t, J=7.5 Hz, 1H), 6.89-6.76 (m, 3H), 5.98 (s, 2H), 4.32 (d, J=5.9 Hz, 2H), 2.80 (s, 3H); MS (ESI(+)) m/e 524 (M+H)⁺.

### EXAMPLE 39

### 3-(2,6-dichlorophenyl)-N-[4-(diethylamino)phenyl]-5-[2-(2-oxopyrrolidin-1-yl)ethyl]isoxazole-4-carboxamide

The titled compound was prepared according to the procedure described in Example 26, substituting 3-oxo-5-(2-oxo-pyrrolidin-1-yl)-pentanoic acid ethyl ester for the 5-dimethylcarbamoyl-3-oxo-pentanoic acid ethyl ester used in Example 26A. ¹HNMR (CDCl₃, 300 MHz), δ 7.69 (br s, 1H, CONH), 7.50-7.38 (m, 3H, Ar—H), 7.19 (d, J=9.15 Hz, 2H, Ar′—H), 6.58 (d, J=9.15 Hz, 2H, Ar′—H), 3.80 (t, 2H, J=6.8 Hz, NCH₂CH₂), 3.48 (t, 2H, J=6.8 Hz, NCH₂CH₂), 3.42 (t, J=7.12 Hz, 2H, CH₂N), 3.30 (q, J=6.80 Hz, 2H, 2 CH₂CH₃), 2.34 (t, J=7.8 Hz, 2H, CH₂CO), 2.01(m, 2H, CH₂), 1.10 (t, J=7.12 Hz, 6H, 2CH₃); MS (ESI) m/e 516.8, 516.1 (M+H)⁺.

### EXAMPLE 40

### 3-(2,6-dichlorophenyl)-N-[4-(diethylamino)phenyl]-5-[2-(3-methyl-2-oxoimidazolidin-1-yl)ethyl]isoxazole-4-carboxamide

The titled compound was prepared according to the procedure described in Example 26, substituting ethyl 2-(3-methyl-2-oxoimidazolidin-1-yl) propionyl acetate for the 5-dimethylcarbamoyl-3-oxo-pentanoic acid ethyl ester used in Example 26A. ¹HNMR (CDCl₃, 300 MHz), δ 8.06 (br s, 1H, CONH), 7.47-7.37 (m, 3H, Ar—H), 7.26 (d, J=9.15 Hz, 2H, Ar′—H), 6.59 (d, J=9.15 Hz, 2H, Ar′—H), 3.72 (t, 2H, J=6.78 Hz, NCH₂CH₂), 3.46 (t, 2H, J=6.78 Hz, NCH₂CH₂), 3.30 (m, 8H, NCH₂CH₂N and NCH₂CH₃), 2.74 (s, 3H, CH₃), 1.10 (t, j=7.12 Hz, 6H, 2CH₃); MS, (ESI) m/e M⁺528.1, 530.0.

### EXAMPLE 41

### 3-(2,6-dichlorophenyl)-N-[4-(diethylamino)phenyl]-5-[2-(dimethylamino)-2-oxoethyl]isoxazole-4-carboxamide

The titled compound was prepared according to the procedure described in Example 26, substituting ethyl 1-(2-N,N-dimethylaminoformylaceto) acetate for the 5-dimethylcarbamoyl-3-oxo-pentanoic acid ethyl ester used in Example 26A. ¹HNMR (CDCl₃, 300 MHz), δ 10.27 (br s, 1H, CONH), 7.45-7.29 (m, 3H, Ar—H), 7.44 (d, J=9.15 Hz, 2H, Ar′—H), 6.60 (d, J=9.15 Hz, 2H, Ar—H), 4.20 (s, 2H, NCH₂), 3.30 (q, J=7.12 Hz, 4H, NCH₂CH₃), 3.32 (s, 6H, CH₃), 3.09 (s, 6H, CH₃), 1.10 (t, J=7.12 Hz, 6H, 2 NCH₂CH₃; MS (ESI) m/e M⁺489.0, 487, (M+H)⁺490.0 530.0.

### EXAMPLE 42

### 3-(2,6-dichlorophenyl)-N-[4-(diethylamino)phenyl]-5-[2-(3,3-dimethyl-2-oxopyrrolidin-1-yl)ethyl]isoxazole-4-carboxamide

The titled compound was prepared according to the procedure described in Example 26, substituting ethyl 3-(3,3-dimethyl-2-oxopyrrolidin-1-yl)-propionyl acetate for the 5-dimethylcarbamoyl-3-oxo-pentanoic acid ethyl ester used in Example 26A. ¹HNMR (CDCl₃, 300 MHz), 6 7.92 (br s, 1H, CONH), 7.48-7.36 (m, 3H, Ar—H), 7.25 (d, J=9.15 Hz, 2H, Ar′—H), 6.59 (d, J=9.15 Hz, 2H, Ar′—H), 3.80 (t, 2H, J=6.44 Hz, NCH₂CH₂), 3.48 (t, 2H, J=6.44 Hz, NCH₂CH₂), 3.33 (t, J=6.78Hz, 2H, CH₂N), 3.30 (q, J=6.80 Hz, 2H, 2 CH₂CH₃), 1.48 (t, J=6.78 Hz, 2H, CH₂), 1.55 (s, 6H, CH₃), 1.10 (s, 6H, CH₃), 1.11 (t, J=7.12, 6H, 2CH₂CH₃); MS (ESI) m/e M⁺543.0, 545.0.

### EXAMPLE 43

### 3-(2,6-dichlorophenyl)-N-[4-(diethylamino)cyclohexyl]-5-methylisoxazole-4-carboxamide

### EXAMPLE 43A

### (4-{[3-(2,6-Dichloro-phenyl)-5-methyl-isoxazole-4-carbonyl]-amino}-cyclohexyl)-carbamic acid tert-butyl ester

The titled compound was prepared according to the procedure described in Example 1, substituting trans-(4-amino-cyclohexyl)-carbamic acid tert-butyl ester (Smith, J.; Liras, J. L.; Schneider, S. E.; Anslyn, E. V. J. Org. Chem. 1996, 61, 8811-8818) for N,N-diethylphenylene diamine. MS (ESI) m/e 466,468 (M−H)⁻.

### EXAMPLE 43B

### 3-(2,6-Dichloro-phenyl)-5-methyl-isoxazole-4-carboxylic acid (4-amino-cyclohexyl)-amide

The titled compound was prepared according to the procedure described in Example 19D, substituting the t-butyl carbamate from Example 43A for the t-butyl carbamate from Example 19C. Light yellow solid; MS (ESI) m/e 366, 368 (M−H)⁻.

### EXAMPLE 43C

### 3-(2,6-dichlorophenyl)-N-[4-(diethylamino)cyclohexyl]-5-methylisoxazole-4-carboxamide

A mixture of 3-(2,6-dichloro-phenyl)-5-methyl-isoxazole-4-carboxylic acid (4-amino-cyclohexyl)-amide HCl salt from Example 43B (84 mg, 0.21 mmol), acetic acid (26 μL, 0.46 mmol), acetaldehyde (29 μL, 0.53 mmol), and Na(OAc)₃BH (131 mg, 0.63 mmol) in 3 mL of 1,2-dichloroethane was stirred for over night at room temperature. The volatile solvent was then removed under reduced pressure, and the crude residue purified by preparative HPLC to provide the titled compound as a light brown solid (57mg, 65% yield). ¹H NMR (300 MHz, DMSO-d₆) δ 7.97 (d, J=7.8 Hz, 1H), 7.60 (d, J=8.8 Hz, 1H), 7.60 (d, J=6.8 Hz, 1H), 7.53 (dd, J=9.5, 6.1 Hz, 1H), 3.64-3.48 (m, 1H), 3.28-3.11 (m, 2H), 3.11-2.98 (m, 2H), 2.63 (s, 3H), 1.97 (br d, J=11.9 Hz, 2H), 1.89 (br d, J=10.9 Hz, 2H), 1.54 (br q, J=11.9 Hz, 2H), 1.33 (br q, J=11.9 Hz, 2H), 1.21 (t, J=7.5 Hz, 6H); MS (ESI) m/e 424, 426, 428 (M+H)⁺.

### EXAMPLE 44

### 3-(2,6-dichlorophenyl)-N-[4-(diethylamino)-2-methylphenyl]-5-[2-(1,3-dioxan-2-yl)ethyl]isoxazole-4-carboxamide

The titled compound was prepared according to the procedure described in Example 27, substituting N,N-diethyl-2-methyl-1,4-phenylenediamine for N,N-diethyl-1,4-phenylenediamine. ¹H NMR (300 MHz, DMSO-d₆) δ 9.16 (s, 1H), 7.63-7.50 (m, 3H), 6.93 (m, 1H), 6.49-6.42 (m, 2H), 4.58 (t, 1H), 4.01-3.95 (m, 2H), 3.73-3.63 (m, 2H), 3.29-3.18 (m, 2H), 2.05 (s, 3H), 2.04-1.28 (m, 4H), 1.05 (t, J=7.1 Hz, 6H). MS (ESI(+)) m/e 532 (M+H)⁺.

### EXAMPLE 45

### 3-(2,6-dichlorophenyl)-N-[4-(diethylamino)phenyl]-5-[2-(1,3-dioxolan-2-yl)ethyl]isoxazole-4-carboxamide

The titled compound was prepared according to the procedure described in Example 26, substituting ethyl 3-(1,3-dioxolan-2-yl)-propionyl acetate for the 5-dimethylcarbamoyl-3-oxo-pentanoic acid ethyl ester used in Example 26A. 1HNMR (CDCl₃, 300 MHz), δ 7.43 (br s, 1H, CONH), 7.50-7.38 (m, 3H, Ar—H), 7.14 (d, J=9.15 Hz, 2H, Ar′—H), 6.58 (d, J=9.15 Hz, 2H, Ar′—H), 5.10 (t, J=4.41 Hz, 1H, CH), 4.01 (t, 2H, J=6.78 Hz, OCH₂CH₂O), 3.89 (t, 2H, J=6.78 Hz, OCH₂CH₂O), 3.35 (t, J=7.46 Hz, 2H, CH₂N), 3.30 (q, J=6.80 Hz, 4H, 2 CH₂CH₃), 2.30 (m, 2H, CH₂CH₂CH), 1.11 (t, J=6.78 Hz, 6H, 2CH₂CH₃); MS (ESI) m/e M⁺504.0, 502.0.

### EXAMPLE 46

### 3-(2,6-dichlorophenyl)-N-[4-(diethylamino)phenyl]-5-(2-methoxyethyl)isoxazole-4-carboxamide

The titled compound was prepared according to the procedure described in Example 26, substituting ethyl 1-(3-methoxypropionyl) acetate for the 5-dimethylcarbamoyl-3-oxo-pentanoic acid ethyl ester used in Example 26A. ¹HNMR (CDCl₃, 300 MHz), δ 9.08 (br s, 1H, CONH), 7.42-7.29 (m, 3H, Ar—H), 7.31 (d, J=9.16 Hz, 2H, Ar′—H), 6.60 (d, J=9.16 Hz, 2H, Ar′—H), 3.90 (t, J=5.76 Hz, 2H, NCH₂CH₂), 3.38 (t, J=5.76 Hz, 2H, NCH₂CH₂), 3.50 (s, 3H, CH₃), 3.30 (q, J=7.12 Hz, 4H, 2 CH₂CH₃), 1.11 (t, J=7.12 Hz, 6H, 2CH₂CH₃); MS (ESI) m/e M⁺462.0, 460.0.

### EXAMPLE 47

### 3-(2,6-dichlorophenyl)-N-[4-(diethylamino)phenyl]-5-(2-tetrahydrofuran-2-ylethyl)isoxazole-4-carboxamide

The titled compound was prepared according to the procedure described in Example 26, substituting 3-oxo-5-(tetrahydro-furan-2-yl)-pentanoic acid ethyl ester for the 5-dimethylcarbamoyl-3-oxo-pentanoic acid ethyl ester used in Example 26A. ¹HNNMR (CDCl₃, 300 MHz), δ 8.81 (br s, 1H, CONH), 7.45-7.30 (m, 3H, Ar—H), 7.28 (d, J=9.16 Hz, 2H, Ar′—H), 6.60 (d, J=9.16 Hz, 2H, Ar′—H), 3.97-3.76 (m, 3H, CHOCH₂), 3.36 (t, J=5.76 Hz, 2H, NCH₂CH₂), 3.19 (t, J=5.76 Hz, 2H, NCH₂CH₂), 3.30 (q, J=7.12 Hz, 4H, 2 CH₂CH₃), 2.22-1.89 (m, 4H, CH₂CH₂), 1.11 (t, J=7.12 Hz, 6H, 2CH₂CH₃0; MS (ESI) m/e (M)⁺500.0, 502.0, (M+H)⁺503.8.

### EXAMPLE 48

### 3-(2,6-dichlorophenyl)-N-[2-(3,4-dihydroisoquinolin-2(1H)-ylcarbonyl)phenyl]-5-methylisoxazole-4-carboxamide

The titled compound was prepared according to the procedure described in Example 38, substituting 1,2,3,4-tetrahydro-isoquinoline for C-benzo[1,3]dioxol-5-yl-methylamine. ¹H NMR (300 MHz, DMSO-d₆) δ 9.72 (s, 1H), 7.65-6.89 (m, 11H), 4.67-4.4 (m, 2H), 3.80-3.40(m, 4H), 2.71 (s, 3H). MS (ESI(+)) m/e 506 (M+H)⁺.

### EXAMPLE 49

### 3-(2,6-dichlorophenyl)-N-(2-{[(2,3-dihydro-1-benzofuran-5-ylmethyl)amino]carbonyl}phenyl)-5-methylisoxazole-4-carboxamide

The titled compound was prepared according to the procedure described in Example 38, substituting C-(2,3-dihydro-benzofuran-5-yl)-methylamine for C-benzo[1,3]dioxol-5-yl-methylamine. ¹H NMR (300 MHz, DMSO-d₆) δ 11.62 (s, 1H), 9.24 (t, J=5.8 Hz, 1H), 8.33 (d, J=8.5 Hz, 1H), 7.75 (d, J=7.8 Hz, 1H), 7.62-7.45 (m, 4H), 7.19-7.00 (m, 3H), 5.98 (s, 2H), 6.70 (d, J=8.1 Hz, 1H), 4.50 (t, J=8.8 Hz, 2H), 4.32 (d, J=6.1 Hz, 2H), 3.14 (t, J=8.8 Hz, 2H), 2.80 (s, 3H). MS (ESI(+)) m/e 522 (M+H)⁺.

### EXAMPLE 50

### 3-(2,6-dichlorophenyl)-N-{(1R)-1-[4-(diethylamino)phenyl]ethyl}-5-methylisoxazole-4-carboxamide

To 3-(2,6-dichloro-phenyl)-5-methyl-isoxazole-4-carboxylic acid (272 mg, 1.0 mmol) in DMF (1.5 mL) was added (+)-(R)-1-(4-Nitro-phenyl)-ethylamine hydrochloride (202 mg, 1.0 mmol), Et₃N (202 mg, 2.0 mmol), and TBTU (321 mg, 1.0 mmol) at 0° C. After 2 hours ice chips and 50 mL ethyl acetate were added. Organic layer was washed with water (3×30 mL), dried over MgSO₄, and then concentrated under reduced pressure. The crude material was dissolved in ethanol (15 mL) and water (1 mL) followed by addition of NH₄Cl (107 mg, 2.0 mmol) and Fe (1.12 g, 20 mmol). The insoluble material was filtered off after 16 hours and the filtrate was dried over MgSO₄ and concentrated under reduced pressure. The crude material was dissolved in methanol buffer (1 M, pH4, 7.5 mL) followed by addition of acetaldehyde (132 mg, 3.0 mmol) and sodium cyanoborohydride (124 mg, 2.0 mmol). The volatiles were removed under reduced pressure after 3 hour and the residue was purified by reversed phase HPLC (5-100% 1% TFA in acetonitrile) to provide the titled compound (60 mg, 13% over 3 steps). ¹H NMR (300 MHz, DMSO-d₆) δ 8.15 (d, J=8.1 Hz, 1H), 7.60 (d, J=9.6 Hz, 1H), 7.59 (d, J=6.0 Hz, 1H), 7.52 (dd, J=6.0, 9.6 Hz, 1H), 7.04 (d, J=8.7 Hz, 2H), 6.57 (d, J=8.7 Hz, 2H), 4.84 (quintet, J=7.2 Hz, 1H), 3.29 (q, J=7.2 Hz, 4H), 2.64 (s, 3H), 1.32 (d, J=7.2 Hz, 3H), and 1.06 (t, J=7.2 Hz, 6H). MS (ESI) positive ion 446 (M+H)^{+,} 448 (M+H)⁺; negative ion 444 (M−H)⁻, 446 (M−H)⁻.

### EXAMPLE 51

### 3-(2,6-dichlorophenyl)-N-[4-(diethylamino)phenyl]-5-r2-(2-oxopiperidin-1-yl)ethyl]isoxazole-4-carboxamide

The titled compound was prepared according to the procedure described in Example 26, substituting 3-oxo-5-(2-oxo-piperidin-1-yl)-pentanoic acid ethyl ester for the 5-dimethylcarbamoyl-3-oxo-pentanoic acid ethyl ester used in Example 26A. ¹HNMR (CDCl₃, 300 MHz), δ 8.19 (br s, 1H, CONH), 7.47-7.35 (m, 3H, Ar—H), 7.30 (d, J=9.16 Hz, 2H, Ar′—H), 6.59 (d, J=9.16 Hz, 2H, Ar′—H), 3.85 (t, J=6.78 Hz, 2H, NCH₂), 3.50 (t, J=6.76 Hz, 2H, NCH₂CH₂), 3.32 (t, J=6.76 Hz, 2H, NCH₂CH₂), 3.30 (q, J=7.12 Hz, 4H, 2 CH₂CH₃), 2.33 (br m, 2H, CH₂CO), 1.75 (br m, 4H, CH₂CH₂), 1.11 (t, J=7.12 Hz, 6H, 2CH₂CH₃), (ESI) m/e (M)⁺529.1, 531.0, (M+H)⁺503.8.

### EXAMPLE 52

### 5-{2-[acetyl(methyl)amino]ethyl}-3-(2,6-dichlorophenyl)-N-[4-(diethylamino)phenyl]isoxazole-4-carboxamide

The titled compound was prepared according to the procedure described in Example 26, substituting 5-(acetyl-methyl-amino)-3-oxo-pentanoic acid ethyl ester for the 5-dimethylcarbamoyl-3-oxo-pentanoic acid ethyl ester used in Example 26A. ¹HNMR (CDCl₃, 300 MHz), δ 7.85 (br s, 1H, CONH), 7.7.56-7.37 (m, 3H, Ar—H), 7.00 (d, J=9.16 Hz, 2H, Ar′—H), 6.57 (d, J=9.16 Hz, 2H, Ar′—H), 3.87 (t, J=6.78 Hz, 2H, N CH₂CH₂), 3.46 (t, J=6.78 Hz, 2H, N CH₂CH₂), 3.30 (q, J=7.12 Hz, 4H, 2 CH₂CH₃), 3.02 (s, 3H, NCH₃), 2.05 (s, 3H, CH₃CO), 1.11 (t, J=7.12 Hz, 6H, 2CH₂CH₃); MS (ESI) m/e M⁺503.0, 501.0.

### EXAMPLE 53

### 3-(2,6-dichlorophenyl)-N-(4-{2-[(3-ethoxypropyl)amino]ethyl}phenyl)-5-methylisoxazole-4-carboxamide

### EXAMPLE 53A

### 3-(2,6-dichlorophenyl)-5-methyl-isoxazole-4-carboxylic acid [4-(2-hydroxy-ethyl)-phenyl]amide

To a stirred solution of 3-(2,6-dichlorophenyl)-5-methylisoxazole-4-carboxylic acid (2.04 g, 7.5 mmol), 4-aminophenethyl alcohol (1.03g, 7.5 mmol), TBTU (2.65 g, 8.25 mmol), and anhydrous DMF (7 mL) was added triethylamine (2.1 mL, 15 mmol). The mixture was stirred for 24 hours, poured into aqueous sodium bicarbonate and extracted with ethyl acetate. The combined organic layers were washed with water and brine, dried over sodium sulfate, filtered and concentrated under reduced pressure. The crude product was purified by silica gel column (40-60% ethyl acetate/hexanes) to provide the titled compound (1.72 g, 58%). ¹H NMR (DMSO): δ 10.07 (s, 1H), 7.51-7.62 (m, 3H), 7.43 (d, 2H), 7.14 (d, 2H), 4.58 (t, 1H), 3.53 (m, 2H), 3.31 (s, 3H), 2.66 (m, 2H). MS (ESI): 391 (M+H)⁺, 389 (M−H)⁻

### EXAMPLE 53B

### 3-(2,6-dichlorophenyl)-5-methyl-N-[4-(2-oxoethyl)phenyl]isoxazole-4-carboxamide

The alcohol from Example 53A (783 mg, 2 mmol) was dissolved in anhydrous methylene chloride (10 mL) and cooled to 0° C. Dess-Martin periodinane (1.02 g, 2.4 mmol) was added, the reaction stirred at 0° C. for 15 minutes, then allowed to warm to room temperature over 2 hours and then concentrated to half volume under reduced pressure. The mixture was purified by silica gel column (50% ethyl acetate/hexane) to provide the titled compound (646 mg, 83%). ¹H NMR (CDCl₃): δ 9.70 (t, 1H), 7.52-7.59 (m, 3H), 7.09-7.20 (m, 4H), 6.9 (br.s, 1H), 3.63 (d, 2H), 2.87 (s, 3H). MS (ESI): 389 (M+H)^{+,} 387 (M−H)⁻

### EXAMPLE 53C

### 3-(2,6-dichlorophenyl)-N-(4-{2-[(3-ethoxypropyl)amino]ethyl}phenyl)-5-methylisoxazole-4-carboxamide

To a stirred solution of 3-(2,6-dichlorophenyl)-5-methyl-N-[4-(2-oxoethyl)phenyl]isoxazole-4-carboxamide from example 53B (97.4 mg, 0.25 mmol), 3-ethoxypropylamine (33 μL, 0.275 mmol) in 1 M sodium acetate/acetic acid buffer (pH 4) in methanol (3 mL) was added sodium cyanoborohydride (31.4 mg, 0.50 mmol). After 24 hours saturated sodium bicarbonate (3 mL) was added and the reaction stirred for 1 hour. The reaction mixture was extracted with ethyl acetate, washed with brine, dried over sodium sulfate, filtered and concentrated under reduced pressure. The crude product was purified by silica gel column (10% methanol/1% ammonium hydroxide/ethyl acetate). The addition of 4 M HCl/dioxane (1 mL) formed the hydrochloride salt which was recrystallized from chloroform/hexanes to provide the titled compound (30.8 mg, 24%). ¹H NMR (DMSO): δ 10.19 (s, 1H), 8.5 (br s, 1H), 7.51-7.63 (m, 5 H), 7.21 (s, 1H), 7.19 (s, 1H), 3.41 (q, 3H), 3.37 (d, 2H), 3.12 (br t, 2H), 2.97 (br t, 2H), 2.86 (br t, 2H), 2.74 (s, 3H), 1.83 (br t, 2H), 1.1 (t, 2H). MS (ESI): 476 (M+H)⁺.

### EXAMPLE 54

### 3-(2,6-dichlorophenyl)-N-(4-{2-[(3-isopropoxypropyl)amino]ethyl}phenyl)-5-methylisoxazole-4-carboxamide

The titled compound was prepared according to the same procedure as described in Example 53, substituting the 3-ethoxypropylamine used in Example 53C with 3-isopropoxypropylamine. Yield: 34.8 mg (26%). ¹H NMR (DMSO): δ 10.19 (s, 1H), 8.49 (br s, 1H), 7.51-7.63 (m, 5H), 7.21 (s, 1H), 7.19 (s, 1H), 3.50 (m, 1H), 3.29 (t, 2H), 3.12 (m, 2 H), 2.97 (m, 2H), 2.89 (m, 2H), 2.73 (s, 3H), 1.80 (m, 2H), 1.06 (t, 6 H). MS (ESI): 490 (M+H)^{+,} 488 (M−H)⁻.

### EXAMPLE 55

### 3-(2,6-dichlorophenyl)-5-methyl-N-(4-{2-[(2-phenoxyethyl)amino]ethyl}phenyl)isoxazole-4-carboxamide

The titled compound was prepared according to the same procedure as in example 53, substituting the 3-ethoxypropylamine used in Example 53C with 2-phenoxyethylamine (36 μL, 0.275 mmol). Yield: 39.7 mg (29%). ¹H NMR (DMSO): δ 10.19 (s, 1H), 8.86 (br s, 1H), 7.52-7.63 (m, 4H), 7.31-7.37 (m, 3H), 7.22 (s, 1H), 7.19 (s, 1H), 6.98-7.02 (m, 3H), 4.23 (m, 2H), 3.38 (br t, 2H), 3.22 (br t, 2H), 2.92 (br t, 2H), 2.73 (s, 3H). MS (ESI): 510 (M+H)⁺, 508 (M−H)⁻.

What is claimed is

1. A compound according to formula (I), 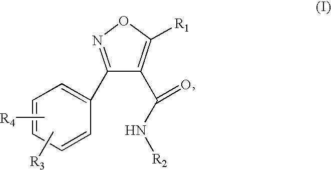
or a therapeutically suitable salt or prodrug thereof, wherein
R₁ is a member selected from the group consisting of alkoxyalkyl, alkyl, alkylC(O)NHalkyl, alkylS(O)₂NHalkyl, alkenyl, aryl, arylalkyl, heterocycle, heterocyclealkyl, hydroxyalkyl, RₐR_{b}N—, RₐR_{b}Nalkyl, and RₐR_{b}Ncarboxyalkyl, wherein the alkyl group of said arylalkyl and the alkyl group of said heterocyclealkyl may be substituted with 0, 1 or 2 groups that are a member selected from the group consisting of halogen and hydroxy;
R₂ is a member selected from the group consisting of alkyl, aryl, arylalkyl, cycloalkyl, cycloalkylalkyl, cycloalkenyl, cycloalkenylalkyl, heterocycle and heterocyclealkyl;
R₃ and R₄ are each members independently selected from the group consisting of hydrogen, alkyl, alkoxy, aryl, halogen, haloalkyl, cycloalkyl, cyano and nitro;
Rₐ and R_{b} are each members independently selected from the group consisting of hydrogen, alkoxyalkyl, alkyl, alkylcarbonyl, alkylsulfonyl, aryloxyalkyl and R_{c}R_{d}Ncarboxyalkylcarbonyl; and
R_{c} and R_{d} are each independently a member selected from the group consisting of hydrogen, and alkyl.

2. A compound according to formula (Ia), 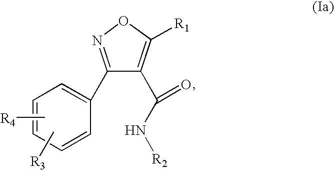
or a therapeutically suitable salt or prodrug thereof, wherein
R₁ is a member selected from the group consisting of aryl, arylalkyl, heterocycle, and heterocyclealkyl, wherein the alkyl group of said arylalkyl and the alkyl group of said heterocyclealkyl may be substituted with 0, 1 or 2 groups that are each members independently selected from the group consisting of halogen and hydroxy;
R₂ is a member selected from the group consisting of alkyl, aryl, arylalkyl, cycloalkyl, cycloalkylalkyl, cycloalkenyl, cycloalkenylalkyl, heterocycle and heterocyclealkyl;
R₃ and R₄ are each members independently selected from the group consisting of hydrogen, alkyl, alkoxy, aryl, halogen, haloalkyl, cycloalkyl, cyano and nitro.

3. The compound according to claim 2, wherein
R₂ is a member selected from the group consisting of aryl, arylalkyl, heterocycle and heterocyclealkyl; and
R₃ and R₄ are each members independently selected from the group consisting of hydrogen, alkyl, halogen and nitro.

4. The compound according to claim 3 that is selected from the group consisting of
3-(2,6-dichlorophenyl)-N-[4-(diethylamino)phenyl]-5-(2-furyl)isoxazole-4-carboxamide;
3-(2,6-dichlorophenyl)-N-[4-(diethylamino)phenyl]-5-(2-phenylethyl)isoxazole-4-carboxamide;
3-(2,6-dichlorophenyl)-N-[4-(diethylamino)phenyl]-5-[2-(1,3-dioxan-2-yl)ethyl]isoxazole-4-carboxamide;
3-(2,6-dichlorophenyl)-N-[4-(diethylamino)phenyl]-5-[3-(1,3-dioxan-2-yl)propyl]isoxazole-4-carboxamide;
3-(2,6-dichlorophenyl)-N-[4-(diethylamino)phenyl]-5-(2-hydroxy-2-phenylethyl)isoxazole-4-carboxamide;
3-(2,6-dichlorophenyl)-N-[4-(diethylamino)phenyl]-5-(2-tetrahydro-2H-pyran-2-ylethyl)isoxazole-4-carboxamide;
3-(2,6-dichlorophenyl)-N-[4-(diethylamino)phenyl]-5-(tetrahydro-2H-pyran-4-ylmethyl)isoxazole-4-carboxamide;
3-(2,6-dichlorophenyl)-5-[2-(1,3-dioxan-2-yl)ethyl]-N-{2-[(2-methyl-1,2,3,4-tetrahydroisoquinolin-1-yl)methyl]phenyl}isoxazole-4-carboxamide;
3-(2,6-dichlorophenyl)-N-[4-(diethylamino)phenyl]-5-[(2-oxopyrrolidin 1-yl)methyl]isoxazole-4-carboxamide;
3-(2,6-dichlorophenyl)-N-[4-(diethylamino)phenyl]-5-[2-(2-oxopyrrolidin-1-yl)ethyl]isoxazole-4-carboxamide;
3-(2,6-dichlorophenyl)-N-[4-(diethylamino)phenyl]-5-[2-(3-methyl-2-oxoimidazolidin-1-yl)ethyl]isoxazole-4-carboxamide;
3-(2,6-dichlorophenyl)-N-[4-(diethylamino)phenyl]-5-[2-(3,3-dimethyl-2-oxopyrrolidin-1-yl)ethyl]isoxazole-4-carboxamide;
3-(2,6-dichlorophenyl)-N-[4-(diethylamino)-2-methylphenyl]-5-[2-(1,3-dioxan-2-yl)ethyl]isoxazole-4-carboxamide;
3-(2,6-dichlorophenyl)-N-[4-(diethylamino)phenyl]-5-[2-(1,3-dioxolan-2-yl)ethyl]isoxazole-4-carboxamide;
3-(2,6-dichlorophenyl)-N-[4-(diethylamino)phenyl]-5-(2-tetrahydrofuran-2-ylethyl)isoxazole-4-carboxamide; and
3-(2,6-dichlorophenyl)-N-[4-(diethylamino)phenyl]-5-[2-(2-oxopiperidin-1-yl)ethyl]isoxazole-4-carboxamide.

5. A compound according to formula (Ib), 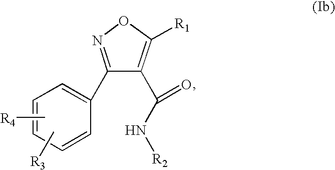
or a therapeutically suitable salt or prodrug thereof, wherein
R₁ is a member selected from the group consisting of alkoxyalkyl, alkyl, alkylC(O)NHalkyl, alkylS(O)₂NHalkyl, alkenyl, hydroxyalkyl, RₐR_{b}N—, RₐR_{b}Nalkyl, and RₐR_{b}Ncarboxyalkyl;
R₂ is a member selected from the group consisting of alkyl, aryl, arylalkyl, cycloalkyl, cycloalkylalkyl, cycloalkenyl, cycloalkenylalkyl, heterocycle and heterocyclealkyl;
R₃ and R₄ are each members independently selected from the group consisting of hydrogen, alkyl, alkoxy, aryl, halogen, haloalkyl, cycloalkyl, cyano and nitro;
Rₐ and R_{b} are each members independently selected from the group consisting of hydrogen, alkoxyalkyl, alkyl, alkylcarbonyl, alkylsulfonyl, aryloxyalkyl and R_{c}R_{d}Ncarboxyalkylcarbonyl; and
R_{c} and R_{d} are each members independently selected from the group consisting of hydrogen, and alkyl.

6. The compound according to claim 5, wherein
R₂ is a member selected from the group consisting of aryl, arylalkyl and cycloalkyl; and
R₃ and R₄ are each members independently selected from the group consisting of hydrogen, alkyl, halogen and nitro.

7. The compound according to claim 6 that is selected from the group consisting of
3-(2,6-dichlorophenyl)-N-[4-(diethylamino)phenyl]-5-methylisoxazole-4-carboxamide;
3-(2-chloro-6-fluorophenyl)-N-[4-(diethylamino)phenyl]-5-methylisoxazole-4-carboxamide;
5-but-3-enyl-3-(2,6-dichlorophenyl)-N-[4-(diethylamino)phenyl]isoxazole-4-carboxamide;
3-(2,6-dichlorophenyl)-N-[4-(diethylamino)phenyl]-5-(3,4-dihydroxybutyl)isoxazole-4-carboxamide;
3-(2,6-dichlorophenyl)-N-[4-(diethylamino)phenyl]-5-ethylisoxazole-4-carboxamide;
3-(2-chloro-6-nitrophenyl)-N-[4-(diethylamino)phenyl]-5-methylisoxazole-4-carboxamide;
3-(2,6-dichlorophenyl)-N-[4-(diethylamino)phenyl]-5-(4-hydroxybutyl)isoxazole-4-carboxamide;
3-(2,6-dichlorophenyl)-N-[4-(diethylamino)-2-methylphenyl]-5-methylisoxazole-4-carboxamide;
N-[4-(diethylamino)phenyl]-5-methyl-3-(2-nitrophenyl)isoxazole-4-carboxamide;
3-(2,6-dichlorophenyl)-N-{4-[ethyl(isopropyl)amino]phenyl}-5-methylisoxazole-4-carboxamide;
5-(4-aminobutyl)-3-(2,6-dichlorophenyl)-N-[4-(diethylamino)phenyl]isoxazole-4-carboxamide;
3-(2-bromophenyl)-N-[4-(diethylamino)phenyl]-5-methylisoxazole-4-carboxamide;
3-(2,6-dichlorophenyl)-N-[4-(diethylamino)phenyl]-5-propylisoxazole-4-carboxamide;
3-(2,6-dichlorophenyl)-N-[4-(diethylamino)phenyl]-5-isopropylisoxazole-4-carboxamide;
3-(2,6-dichlorophenyl)-N-[4-(diethylamino)-2-methoxyphenyl]-5-methylisoxazole-4-carboxamide;
N-{4-[tert-butyl(ethyl)amino]phenyl}-3-(2,6-dichlorophenyl)-5-methylisoxazole-4-carboxamide;
3-(2,6-dichlorophenyl)-N-[4-(diethylamino)-2-hydroxyphenyl]-5-methylisoxazole-4-carboxamide;
N-{4-[(2-chloroethyl)(ethyl)amino]phenyl}-3-(2,6-dichlorophenyl)-5-methylisoxazole-4-carboxamide;
3-(2,6-dichlorophenyl)-N-{4-[ethyl(propyl)amino]phenyl}-5-methylisoxazole-4-carboxamide;
N-{4-[butyl(ethyl)amino]phenyl}-3-(2,6-dichlorophenyl)-5-methylisoxazole-4-carboxamide;
3-(2,6-dichlorophenyl)-N-[4-(diethylamino)-2-methylphenyl]-5-propylisoxazole-4-carboxamide;
3-(2,6-dichlorophenyl)-N-[4-(diethylamino)-2-(piperidin-1-ylcarbonyl)phenyl]-5-methylisoxazole-4-carboxamide;
3-(2,6-dichlorophenyl)-N-[4-(diethylamino)-2-ethylphenyl]-5-methylisoxazole-4-carboxamide;
3-(2,6-dichlorophenyl)-N-[4-(diethylamino)phenyl]-5-[3-(dimethylamino)-3-oxopropyl]isoxazole-4-carboxamide;
5-[4-(acetylamino)butyl]-3-(2,6-dichlorophenyl)-N-[4-(diethylamino)phenyl]isoxazole-4-carboxamide;
3-(2,6-dichlorophenyl)-N-[4-(diethylamino)phenyl]-5-{4-[(methylsulfonyl)amino]butyl}isoxazole-4-carboxamide;
3-(2,6-dichlorophenyl)-N-[4-(diethylamino)phenyl]-5-(3-hydroxypropyl)isoxazole-4-carboxamide;
5-butyl-3-(2,6-dichlorophenyl)-N-[4-(diethylamino)phenyl]isoxazole-4-carboxamide;
N-(2-{[(1,3-benzodioxol-5-ylmethyl)amino]carbonyl}phenyl)-3-(2,6-dichlorophenyl)-5-methylisoxazole-4-carboxamide;
3-(2,6-dichlorophenyl)-N-[4-(diethylamino)phenyl]-5-[2-(dimethylamino)-2-oxoethyl]isoxazole-4-carboxamide;
3-(2,6-dichlorophenyl)-N-[4-(diethylamino)cyclohexyl]-5-methylisoxazole-4-carboxamide;
3-(2,6-dichlorophenyl)-N-[4-(diethylamino)phenyl]-5-(2-methoxyethyl)isoxazole-4-carboxamide;
3-(2,6-dichlorophenyl)-N-[2-(3,4-dihydroisoquinolin-2(1H)-ylcarbonyl)phenyl]-5-methylisoxazole-4-carboxamide;
3-(2,6-dichlorophenyl)-N-(2-{[(2,3-dihydro-1-benzofuran-5-ylmethyl)amino]carbonyl}phenyl)-5-methylisoxazole-4-carboxamide;
3-(2,6-dichlorophenyl)-N-{(1R)-1-[4-(diethylamino)phenyl]ethyl}-5-methylisoxazole-4-carboxamide;
5-{2-[acetyl(methyl)amino]ethyl}-3-(2,6-dichlorophenyl)-N-[4-(diethylamino)phenyl]isoxazole-4-carboxamide;
3-(2,6-dichlorophenyl)-N-(4-{2-[(3-ethoxypropyl)amino]ethyl}phenyl)-5-methylisoxazole-4-carboxamide;
3-(2,6-dichlorophenyl)-N-(4-{2-[(3-isopropoxypropyl)amino]ethyl}phenyl)-5-methylisoxazole-4-carboxamide; and
3-(2,6-dichlorophenyl)-5-methyl-N-(4-{2-[(2-phenoxyethyl)amino]ethyl}phenyl)isoxazole-4-carboxamide.

8. A method of treating disorders regulated by ghrelin including anorexia, cancer cachexia, eating disorders, age-related decline in body composition, weight gain, obesity, and diabetes mellitus.

9. A pharmaceutical composition comprising a therapeutically effective amount of a compound of the present invention in combination with a pharmaceutically suitable carrier.
